(19) United States
(12) Patent Application Publication
(10) Pub. No.: US 2025/0384556 A1
(43) Pub. Date: Dec. 18, 2025

(54) AUTOMATIC QUALITY CONTROL OF IMAGE DIFFUSION PROCESSING

(71) Applicant: Imeka Solutions Inc., Sherbrooke (CA)

(72) Inventors: Pierre-Marc Jodoin, Eastman (CA); Gabrielle Grenier, Mcmasterville (CA); Loïka Maltais, Sherbrooke (CA); Jean-Christophe Houde, Sherbrooke (CA); Guillaume Theaud, Sherbrooke (CA)

(21) Appl. No.: 19/237,634

(22) Filed: Jun. 13, 2025

Related U.S. Application Data
(60) Provisional application No. 63/661,006, filed on Jun. 17, 2024.

(51) Int. Cl. G06T 7/00; A61B 5/00; G01R 33/563; G01R 33/565; G06T 5/73; G06T 7/11; G06T 7/174; G06T 7/246; G16H 30/40

(52) U.S. Cl. CPC G06T 7/0014; A61B 5/0042; G01R 33/56341; G01R 33/56509; G06T 5/73; G06T 7/11; G06T 7/174; G06T 7/246; G16H 30/40; G06T 2207/10088; G06T 2207/20084; G06T 2207/30016; G06T 2207/30168

(57) ABSTRACT

A computer system that performs quality control (QC) on images associated with diffusion and structural magnetic resonance imaging (MRI) is described. This computer may include: a computation device that executes program instructions; and memory that stores the program instructions. During operation, the computer system may automatically perform a set of validation operations, where, when one or more of the validation operations fails, the images are rejected. Moreover, the set of validation operations may include: performing QC on brain-tissue segmentation; performing QC on diffusion MRI processing; and performing QC on bundles determined from the images using a tractometry technique.

### CROSS REFERENCE TO RELATED APPLICATIONS

This application claims priority under 35 U.S.C. 119(e) to: U.S. Provisional Application Ser. No. 63/661,006, “Automatic Quality Control of Image Diffusion Processing,” filed on Jun. 17, 2024, by Pierre-Marc Jodoin, et al, the contents of which are herein incorporated by reference.

### FIELD

The described embodiments relate to processing of medical images. Notably, the described embodiments relate to techniques for quality control (QC) during processing of medical images to determine diffusion.

### BACKGROUND

The central nervous system consists of the brain and the spinal cord, which each include grey matter and white matter. Grey matter consists primarily of neuronal cell bodies and controls muscular and sensory activity, attention, memory, thought, emotions and, more generally, the processing of information. White matter mainly consists of myelinated axons (which are sometimes referred to as ‘tracts’) that are arranged in bundles, which connect various grey matter areas (the locations of neuronal cell bodies) of the brain to each other, and carry nerve impulses between neurons. Myelin acts as an insulator, which allows electrical signals to jump, rather than course through an axon, thereby increasing the speed of transmission of nerve signals. While previously thought to be passive tissue, white matter affects learning and brain functions, modulates the distribution of action potentials, acts as a relay, and coordinates communication between different brain regions.

White matter is known or suspected to play a role in many neurodegenerative diseases, such as: multiple sclerosis, Alzheimer's disease, Parkinson's disease, amyotrophic lateral sclerosis, traumatic brain injury, alcohol-use disorders, etc. For example, multiple sclerosis is an inflammatory demyelinating disease of the central nervous system that affects white matter. In multiple sclerosis lesions, the myelin sheath around the white-matter axons is deteriorated by inflammation.

In principle, the study of white matter and, thus, understanding of neurodegenerative diseases have been advanced by improved imaging technology, such as diffusion magnetic resonance imaging (dMRI). However, in practice it remains difficult to analyze medical-imaging data to quantify white-matter microstructure and its deterioration because of neurodegenerative diseases.

### SUMMARY

A computer system that performs QC on images associated with diffusion and structural MRI is described. This computer may include: a computation device (such as a processor, a graphics processing unit or GPU, etc.) that executes program instructions; and memory that stores the program instructions. During operation, the computer system automatically performs a set of validation operations (which may be performed sequentially), where, when one or more of the validation operations fails, the images are rejected. Moreover, the set of validation operations include: performing QC on brain-tissue segmentation; performing QC on dMRI processing; and performing QC on bundles determined from the images using a tractometry technique.

Note that the QC on the brain-tissue segmentation may include validating: a volume; a shape; a position; a comparison with a reference-atlas coordinate system (such as a Montreal Neurological Institute or MNI coordinate system); and/or detecting holes in a mask.

Moreover, the QC on the dMRI processing may include validating: a range of diffusion tensor imaging (DTI) metrics; a range of high angular resolution diffusion imaging (HARDI) metrics; and/or ranges of dMRI signals for an orientation distribution function (ODF) and a fiber ODF (fODF).

Furthermore, the QC on the bundles may include validating: streamline statistics; bundle volume; bundle shape; and/or model comparisons.

In some embodiments, the set of validation operations may include: validating metadata associated with the images; performing QC on a diffusion gradient; and/or performing QC on dMRI artifact correction. Note that the diffusion gradient may include: b-values, gradient sampling and/or a gradient configuration. Moreover, the dMRI artifact correction may include: motion detection, eddy-current-distribution detection and/or slice-outlier detection.

Furthermore, the set of validation operations may be implemented using a feed-forward pipeline.

Additionally, the brain-tissue segmentation may include: segmenting the gray matter, gray matter sub-regions, the white matter, white matter sub-regions, the cerebrospinal fluid, and/or deep nuclei regions; and removing voxels that are not associated with brain tissue (such as eyes, bone, mouth, skin, etc.). In some embodiments, the computer system may use the images associated with the structural MRI to extract the brain tissue. Then, using a remainder of the images associated with the dMRI, the computer system may determine local models for voxels, where the local models indicate directions of water diffusion. Note that the computer system may: compute diffusion metrics based at least in part on the local models; recover tracts in the brain tissue; and/or connect grey-matter regions using the bundles.

Moreover, the tractometry technique may include a pretrained neural network, and the computer system may determine the bundles using the pretrained neural network.

Furthermore, a given validation operation may include comparing the given validation operation with a given threshold associated with the given validation operation.

Another embodiment provides a computer for use, e.g., in the computer system.

Another embodiment provides a computer-readable storage medium for use with the computer or the computer system. When executed by the computer or the computer system, this computer-readable storage medium causes the computer or the computer system to perform at least some of the aforementioned operations.

Another embodiment provides a method, which may be performed by the computer or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a drawing of an example of white-matter microstructure in accordance with an embodiment of the present disclosure.

FIGS. 2A and 2B are drawings of examples of q-spaces in accordance with an embodiment of the present disclosure.

FIG. 3 is an image of examples of diffusion metrics that can be derived from a diffusion tensor in accordance with an embodiment of the present disclosure.

FIGS. 4A and 4B are images of examples of two water diffusion models in accordance with an embodiment of the present disclosure.

FIGS. 5A and 5B are images with examples of tractography in accordance with an embodiment of the present disclosure.

FIGS. 6A and 6B are images of example bundles in accordance with an embodiment of the present disclosure.

FIG. 7 is a drawing of an example of an end-to-end tractometry/tractography pipeline in accordance with an embodiment of the present disclosure.

FIG. 8 is an image of an example of a healthy control (HC) and an example of an Alzheimer's Disease (AD) brain in accordance with an embodiment of the present disclosure.

FIG. 9 is an image of slices of an example of an MRI T₁-weighted image of an Alzheimer's disease patient with overlaid dMRI free-water hyperintensities in accordance with an embodiment of the present disclosure.

FIG. 10 is a block diagram illustrating an example of a computer system in accordance with an embodiment of the present disclosure.

FIG. 11 is a flow diagram illustrating an example of a method for providing feedback information using a computer system in FIG. 10 in accordance with an embodiment of the present disclosure.

FIG. 12 is a drawing illustrating an example of communication between components in a computer system in FIG. 10 in accordance with an embodiment of the present disclosure.

FIGS. 13-15 are drawings illustrating examples of an analysis pipeline in accordance with an embodiment of the present disclosure.

FIG. 16 is a drawing illustrating an example of quality control (QC) in an analysis pipeline in accordance with an embodiment of the present disclosure.

FIG. 17 is a drawing illustrating an example of determination of good versus bad quality using a gradient distribution assessment during the QC in FIG. 16 in accordance with an embodiment of the present disclosure.

FIG. 18 is a drawing illustrating an example of self-supervised training of diffusion magnetic resonance imaging (dMRI) harmonization in an analysis pipeline in accordance with an embodiment of the present disclosure.

FIG. 19 is a drawing illustrating an example of dMRI harmonization in an analysis pipeline in accordance with an embodiment of the present disclosure.

FIG. 20 is a drawing illustrating an example of conversion of low-resolution dMRI images into white-matter maps in an analysis pipeline in accordance with an embodiment of the present disclosure.

FIGS. 21-22 are images illustrating examples of tractography in accordance with an embodiment of the present disclosure.

FIGS. 23-29 are drawings illustrating examples of a cleaning and bundling operation in accordance with an embodiment of the present disclosure.

FIG. 30 is a drawing illustrating an example of bundling results in different neurological anatomical regions in accordance with an embodiment of the present disclosure.

FIG. 31 is a drawing illustrating an example of a skull stripping neural network configuration in an analysis pipeline in accordance with an embodiment of the present disclosure.

FIG. 32 is a drawing illustrating an example of a structural MRI segmentation neural network configuration in an analysis pipeline in accordance with an embodiment of the present disclosure.

FIG. 33 is a drawing illustrating an example of a dMRI segmentation neural network configuration in an analysis pipeline in accordance with an embodiment of the present disclosure.

FIG. 34A is an image of white-matter axons in an animal study for healthy controls and Alzheimer's disease on histology illustrating free water and neuroinflammation.

FIG. 34B is an image of white-matter axons in an animal study for healthy controls and Alzheimer's disease illustrating axon myelin degradation.

FIG. 34C is an image of white-matter axons in an animal study for healthy controls and Alzheimer's disease illustrating axon swelling.

FIG. 35 is a drawing illustrating white-matter disease progression from a healthy control, to early/late mild cognitive impairment, and then to Alzheimer's disease.

FIG. 36 is a drawing illustrating an example of free water in white-matter bundles in three groups of subjects, including healthy controls, mild cognitive impairment and Alzheimer's disease.

FIG. 37 is a drawing illustrating an example of free-water change as a function of time in white-matter bundles in the three groups of subjects.

FIG. 38 is a drawing illustrating an example of correlation between free water and age.

FIG. 39A-C are drawings illustrating examples of associations between free water and cognitive tests.

FIG. 40 is a drawing illustrating an example of apparent fiber density in the three groups of subjects.

FIG. 41 is a drawing illustrating an example of a drug that increases the apparent fiber density of a patient.

FIG. 42 is a drawing illustrating an example of associations between apparent fiber density and cerebrospinal-fluid inflammatory markers.

FIG. 43 is a flow chart illustrating an example of a method for automatically performing a set of validation operations in accordance with an embodiment of the present disclosure.

FIG. 44 is a drawing illustrating an example of communication between components in a computer system in FIG. 10 in accordance with an embodiment of the present disclosure.

FIG. 45 is a flow chart illustrating an example of an automatic quality-control (QC) method in accordance with an embodiment of the present disclosure.

FIG. 46 is an image illustrating examples of a T₁ image with an appropriate resolution and an incorrect T₁ image with a low voxel resolution along the z axis in accordance with an embodiment of the present disclosure.

FIG. 47 is a drawing illustrating examples of two different single-shell diffusion MRI configurations in accordance with an embodiment of the present disclosure.

FIG. 48 is an image illustrating examples of a correct b-vector encoding and two incorrect b-vector encodings in accordance with an embodiment of the present disclosure.

FIG. 49 is an image illustrating examples of dMRI artifacts in accordance with an embodiment of the present disclosure.

FIG. 50 is an image illustrating examples of a correct brain-segmentation map and an incorrect-brain segmentation map in accordance with an embodiment of the present disclosure.

FIG. 51 is an image illustrating examples of correct bundles and incorrect (too small) bundles in accordance with an embodiment of the present disclosure.

FIG. 52 is a drawing illustrating an example of bundle QC in the automatic QC method of FIG. 45 in accordance with an embodiment of the present disclosure.

FIG. 53 is a drawing illustrating an example of bundle tractometry QC in the automatic QC method of FIG. 45 in accordance with an embodiment of the present disclosure.

FIG. 54 is a block diagram illustrating an example of a computer in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system that performs QC on images associated with diffusion and structural MRI is described. This computer may include: a computation device (such as a processor, a graphics processing unit or GPU, etc.) that executes program instructions; and memory that stores the program instructions. During operation, the computer system may automatically perform a set of validation operations (which may be performed sequentially), where, when one or more of the validation operations fails, the images are rejected. Moreover, the set of validation operations may include: performing QC on brain-tissue segmentation; performing QC on dMRI processing; and performing QC on bundles determined from the images using a tractometry technique.

By performing the set of validation operations, these QC techniques may address the problems associated with existing brain white-matter analysis techniques. Notably, the QC techniques may ensure high-quality images (such as images associated with MRI) for use in subsequent analysis, such as determining of diffusion. Consequently, the QC techniques may improve the accuracy and relevance of information determined from the images, which may provide improved diagnosis, tracking of disease progression and treatment. Moreover, the determined information (such as a set of white-matter disease biomarkers) may enable further understanding of the diseases and their progression, and may facilitate the development of new treatments.

In the discussion that follows, the analysis and QC techniques are used to analyze dMRI data. However, the analysis and QC techniques may be used to analyze a wide variety of types of magnetic-resonance images (which may or may not involve MRI, e.g., free-induction-decay measurements), such as: magnetic resonance spectroscopy (MRS) with one or more types of nuclei, magnetic resonance spectral imaging (MRSI), magnetic resonance elastography (MRE), magnetic resonance thermometry (MRT), magnetic-field relaxometry and/or another magnetic resonance technique (e.g., functional MRI, metabolic imaging, molecular imaging, blood-flow imaging, diffusion-tensor imaging, etc.). More generally (and provided that the result neurological fibers are in the same reference as the analyzed images), the analysis and QC techniques may be used to analyze measurement results from a wide variety of invasive and non-invasive imaging techniques, such as: X-ray measurements (such as X-ray imaging, X-ray diffraction or computed tomography at one or more wavelengths between 0.01 and 10 nm), neutron measurements (neutron diffraction), electron measurements (such as electron microscopy or electron spin resonance), optical measurements (such as optical imaging or optical spectroscopy that determines a complex index of refraction at one or more visible wavelengths between 300 and 800 nm or ultraviolet wavelengths between 10 and 400 nm), infrared measurements (such as infrared imaging or infrared spectroscopy that determines a complex index of refraction at one or more wavelengths between 700 nm and 1 mm), ultrasound measurements (such as ultrasound imaging in an ultrasound band of wavelengths between 0.2 and 1.9 mm), proton measurements (such as proton scattering), positron emission spectroscopy, positron emission tomography (PET), impedance measurements (such as electrical impedance at DC and/or an AC frequency) and/or susceptibility measurements (such as magnetic susceptibility at DC and/or an AC frequency).

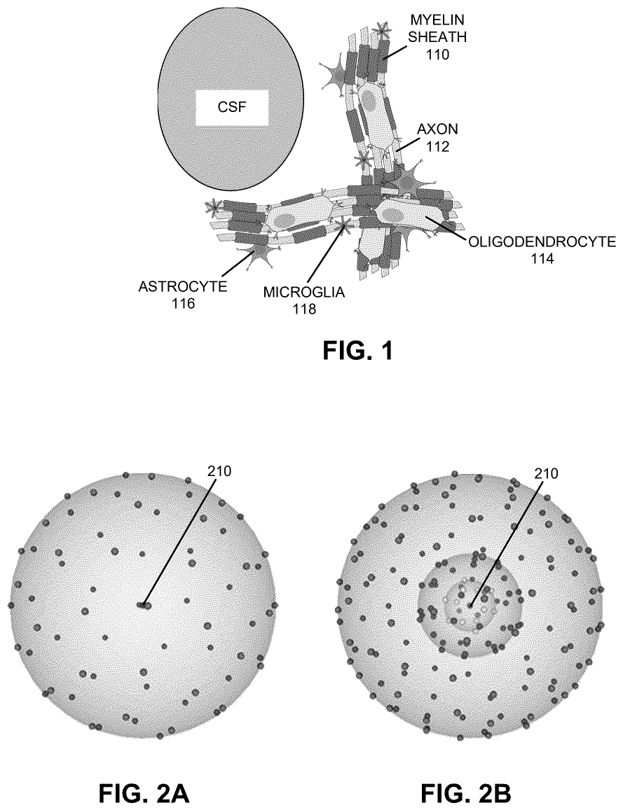

We now describe embodiments of the analysis and QC techniques. The brain is composed of three main regions: the gray matter (GM), the white matter (WM) and cerebrospinal fluid (CSF). As shown in FIG. 1, which presents a drawing of examples of white-matter microstructure, the white matter is composed of axons and glial cells (such as astrocytes, microglias, oligodendrocytes, etc.), which are surrounded by water. An axon is a myelinated and elongated portion of a nerve cell that conducts electric pulses between cortical regions. Axons are organized in fibers, which are themselves grouped into bundles that one can picture as large brain connection pathways. In FIG. 1, note that the myelin sheaths (such as myelin sheath 110) are wrapped around axons (such as axon 112). Moreover, the glial cells include oligodendrocyte 114, astrocyte 116 and microglia 118. All the white-matter components are surrounded by cerebrospinal fluid.

With recent advances in imaging technologies, it is possible to measure the connectivity of the brain and quantify white-matter microstructure, as well as its deterioration, typically due to aging or some neurodegenerative disease. Thanks to the fibrous nature of axons, the diffusion of molecules in the white matter is anisotropic and can be measured by a non-invasive technology, such as dMRI.

Diffusion imaging is the process by which the average displacement of water molecules within a voxel (or volume element, which is sometimes referred to as a ‘voxel’) is measured. A dMRI acquisition protocol is typically designed to acquire a series of diffusion weighted images that are each sensitized to the water diffusion into a particular direction. These images are usually acquired following the seminal pulse gradient spin echo protocol (or a slight variation of it). In an MRI scanner, a strong permanent magnetic field combined with the interplay of radio-frequency pulses and diffusion-sensitizing gradients (which are referred to as ‘pulsed field gradients’ or PFG) forces water molecules to diffuse along a given direction and return a signal back to the MRI scanner.

Each PFG has a magnitude or strength (which is typically called b-value) and a unique pre-defined orientation (which is typically called b-vector). Mathematically, this amounts to G=∥G∥·u, where G is the gradient, ∥G∥ is the norm of that vector (the b-value) and u is a unit vector (the b-vector). An arbitrary large number of diffusion weighted images can be acquired, each with a different PFG b-value/b-vector pair. As such, b-values and b-vectors can be illustrated in a so-called 3D q-space. This is shown in FIGS. 2A and 2B, which present drawings of examples of q-spaces. Notably, if the diffusion weighted images were acquired with the same PFG b-value but different b-vectors, the PFGs can be illustrated as points lying on a sphere whose distance to the origin equals the PFG b-value. This configuration is called single-shell (FIG. 2A). If, however, different b-values are used, the PFG points may lie on different concentric spheres in a configuration called multi-shell (FIG. 2B). While these q-space sampling techniques are often used, other q-space configurations may occur, such as: spectrum sampling, random sampling, grid sampling, and sparse sampling. In FIGS. 2A and 2B, note that dot 210 at the center of the spheres indicates the b0 image, which amounts to a b-value of zero. The b0 image is usually a low-resolution T₂-weighted image that takes into account tissue contrasts and signals in the absence of diffusion gradients. The b0 serves as an anatomical baseline image and is often used to normalize diffusion weighted images.

Thus, diffusion images encode how water molecules diffuse along certain directions. Depending on the environment surrounding a water molecule (such as fat, muscle, fibrous tissues, free water, etc.) it may move more or less freely and, therefore, may return a more or less intense signal. By sampling the q-space with different PFG b-values and b-vectors, a voxel may get different signal values, which can be combined into a compact mathematical model that represents the underlying local tissue structure.

The accuracy of such a local model typically depends heavily on the total number of acquired diffusion weighted images. The simplest model is one derived from a single diffusion acquisition (or one point in the q-space). This results in a raw diffusion-weighted image whose voxel's intensity is proportional to the level of sensitization along one direction. While this simple acquisition can be used for clinical applications (thanks to its simplicity and the short time, e.g., a few minutes, it takes to acquire it), it often has limitations. Notably, given that the measured signal S after the PFG is given by

$$S = {S}_{0} {e}^{- bD} ,$$

where S₀ is the magnetic resonance (MR) signal at baseline, b is the b-value and D is the diffusion coefficient. Therefore, the acquired image may include the raw signal S, but not the diffusion coefficient, per se. Also, because the term e^{-bD} typically behaves very much like the T₂-weighting term e^{-TE/T2} (where TE is the echo time and T₂ the transverse relaxation time), the measured value S is usually heavily affected by relaxation times. This results in variations in the image intensities that can be hard to interpret. In addition, a diffusion image usually has a fairly low signal-to-noise ratio (SNR), which makes it difficult to distinguish the underlying anatomy.

In order to compensate for the T₂ contamination and the low SNR, more diffusion-weighted images usually need to be acquired. Consequently, if two diffusion images with different b-values are acquired and combined, the T₂ contamination can be cancelled out and the diffusion coefficient D (which is often called the ‘apparent diffusion coefficient image’ or ADC image) can be recovered:

$$ADC = D = \frac{ln ( \frac{{s}_{2}}{{s}_{1}} )}{b 1 - b 2} ,$$

where S₁ and S₂ are signal intensities obtained with b1 and b2 b-values. Note that if one of the two signals is obtained with a b0, the following ADC map (which is also called the attenuation map) is obtained:

$$ADC = \frac{ln ( \frac{{s}_{0}}{{s}_{1}} )}{b 1} . \qquad ( 1 )$$

ADC maps are widely used and have been shown to help diagnose brain strokes.

Moreover, in order to increase the SNR, three diffusion images may be acquired. A typical approach is to use the same b-value for all three images, but with b-vectors aligned on the X, Y and Z canonical axis:

$${S}_{x} = {S}_{o} {e}^{- {bD}_{xx ,}} ,$$

$${S}_{y} = {S}_{o} {e}^{- {bD}_{yy}} , and$$

$${S}_{z} = {S}_{o} {e}^{- {bD}_{zz}} ,$$

where Dₓₓ, D_{yy}, D_{zz} are the diffusion coefficients along the three axes. The combination of these images results in the so-called trace DW image (which is sometimes referred to as the short DWI). The combination may be implemented as follows:

$${S}_{DWI} = \sqrt[3]{{S}_{x} {S}_{y} {S}_{z}} = {S}_{o} {e}^{- b ( {D}_{xx} + {D}_{yY} + {D}_{zz} ) / 3} = {S}_{0} {e}^{- b ( {D}_{trace ) / 3}} . \qquad ( 2 )$$

Note that D_{trace} is the trace of the diffusion tensor.

Compared to a single diffusion image S, S_{DWI} typically offers significantly improved contrast. In order to remove the T₂ contamination, a b0 image may be acquired and an attenuated ADC map may be computed following Eqn. 1:

$$ADC = \frac{ln ( \frac{{S}_{0}}{{S}_{DWJ}} )}{b} = \frac{{D}_{trace}}{3} . \qquad ( 3 )$$

Note that, by their very nature, the signal intensities of an ADC map are opposite to those of a DWI, which can be confusing. In order to see this, contemplate from Eqn. 2 that S_{DWI} is maximum when D_{trace} is 0 and decreases exponentially fast as D_{trace} is increasingly positive. This is the exact opposite with the ADC map of Eqn. 3, which is minimum when D_{trace} is 0. Thus, tissues with large diffusivity values (such as free water and cerebrospinal fluid) usually appear bright in an ADC map, while tissues with low diffusivity values (such as stroke and fibrous tissues) typically appear dark. These contrasts are the exact opposite in a DWI image. In order to alleviate the confusion, an exponential ADC image (which is sometimes referred to as a Stejskal-Tanner attenuation map) may be computed:

$${S}_{exp} = \frac{{S}_{DWI}}{{S}_{0}} = {e}^{- b \cdot ADC} . \qquad ( 4 )$$

By acquiring more diffusion images, the 3D structure of the brain tissue may be measured. As discussed previously, the fibrous nature of axons makes the white matter a highly anisotropic environment, with diffusion coefficients larger along the axonal tracts than perpendicular to the tracts. One way of recovering the 3D microstructure of the white matter is by acquiring at least seven images, such as six diffusion-weighted images from six different b-vectors (S₁ to S₆) plus a b0 image (S₀) to recover the unimodal model that is referred to as the diffusion tensor. The diffusion tensor is a 3×3 symmetric positive definite matrix:

$$( {D}_{xx} {D}_{xy} {D}_{xz} {D}_{yx} {D}_{yy} {D}_{yz} {D}_{zx} {D}_{zy} {D}_{zz} ) . \qquad ( 5 )$$

From this matrix, the ADC can be represented as D(u)=u^{T}Du, where u is a b-vector and the b0 attenuated signal is

$${S}_{u} = {e}^{- b \cdot ( {u}^{T} Du )} .$$

Because D has six independent variables, it can be recovered from six (or more) diffusion-weighted images using least-square regression, weighted least-square regression or another technique involving Rician noise.

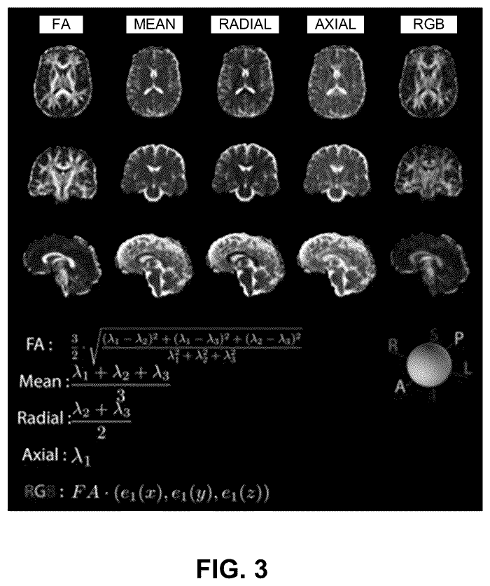

Moreover, from the diffusion tensor D, one can extract three eigenvalues (λ₁, λ₂ and λ₃) whose combination leads to diffusion metrics, such as the fractional anisotropy (FA), the mean diffusivity (MD), the radial diffusivity (RD) and the axial diffusivity (AD), where A, λ₂ and λ₃ are the eigenvalues of the diffusion tensor. These diffusion metrics are shown in FIG. 3, which presents examples of four diffusion metrics that can be derived from a 3×3 diffusion tensor. Because each voxel is associated with a diffusion tensor, note that these diffusion metrics can be visualized as image modalities. Fractional anisotropy is often useful for the assessment of children's brain maturation, dyslexia, and for some psychiatric disorders.

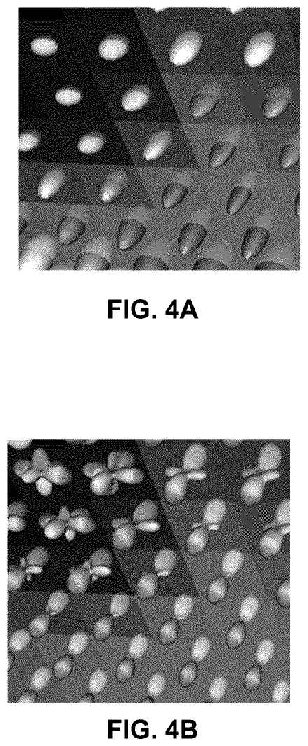

One challenge associated with the diffusion tensor D is that it only encodes one water diffusion direction. Considering that more than two-thirds of the white matter voxels contain crossing brain fibers oriented in more than one direction, this model is often regarded as ill-suited for many applications. As a solution, multidirectional local models have been proposed, such as the bi-tensor and multi-tensor models. Other models account for higher orders by using spherical deconvolution (SD) approaches. In these cases, instead of assuming a fixed number of local orientations, the SD models approximate the local white-matter structure using so-called ODF models or fiber ODF. FIGS. 4A and 4B present images of examples of two local water diffusion models. Notably, FIG. 4A presents a unidirectional shape of a diffusion tensor, and FIG. 4B presents a multidirectional shape of an fODF model. Note that the SD approaches express the acquired diffusion signal in each voxel as a spherical convolution between an ODF and a low-pass filter called a fiber response function (FRF) that describes the signal profile of the white matter. Deconvoluting the measured signal with a FRF leads to the desired fODF.

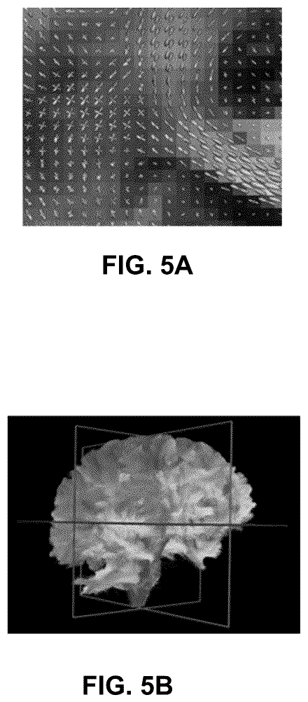

FIGS. 5A and 5B present images of examples of tractography. Tractography is a process in which fibers are reconstructed by iteratively following the local water diffusion models. As shown in FIG. 5A, when a local diffusion model is put on each white matter voxel, the underlying fibrous structure of the white matter tissues may be determined. Notably, FIG. 5A shows a voxel-wise fODF diffusion model overlaid on top of a T₁ image. Moreover, with these models, several tractography techniques have been proposed to reconstruct brain fibers. When enough fibers have been reconstructed, so that each voxel is traversed by at least one fiber, one obtains a so-called whole brain tractogram. This is shown in FIG. 5B, which presents a whole brain tractogram containing 200,000 white matter fibers.

While some global tractography methods have been proposed, typical approaches involve a local iterative technique that builds fibers point by point. The local tractography techniques can be deterministic or probabilistic, may use particle filtering (such as a Particle Filtering Tractography or PFT technique), may be driven by a pre-computed cortex manifold (such as a Surface Enhanced Tracking or SET technique), and/or may be specific to pre-segmented white matter regions (such as the Bundle Specific Tracking or BST technique).

In general, all tracking techniques have limitations, because they all produce spurious or anatomically implausible brain fibers. Such fibers may be too short or too long, have a loopy shape, pass-through non-white matter regions, connect inappropriate gray matter regions and/or connect no gray matter regions at all. These fibers are typically filtered out in a post-processing operation. The remaining anatomically-plausible fibers may then be grouped into fiber bundles.

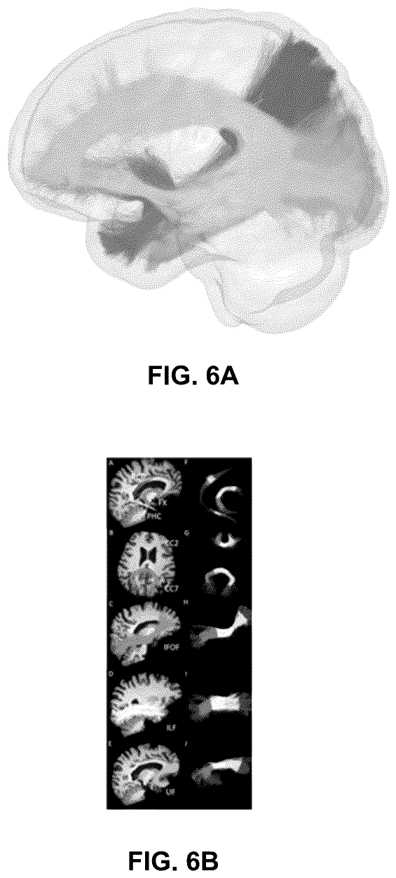

Fibers in a bundle have roughly the same shape and connect the same gray matter regions. The most obvious way to extract white-matter bundles from a whole brain tractogram is by asking a neuroanatomist to manually dissect bundles of interest. However, manual dissecting bundles is long, tedious, and prone to large inter- and intra-expert variability. Automated techniques, such as QuickBundles (QB), QuickBundlesX (QBx), Deep Fiber Clustering (DFC), RecoBundles (RB), RecoBundlesX (RBx), TractSeg, XTRACT, FINTA, FIESTA, Deep White Matter Analysis (WMA), and BINTA, have been proposed to accelerate and increase the reproducibility of this process. FIGS. 6A and 6B presents images of examples of bundles. Notably, FIG. 6A shows five fiber bundles obtained by fiber clustering: arcuate fasciculus, uncinate fasciculus, inferior fronto-occipital fasciculus, splenium of the corpus callosum, and inferior longitudinal fasciculus. Moreover, FIG. 6B shows five bundles divided into sub-regions (from red to blue).

These bundles can be useful to assess the integrity of the white matter into specific regions associated with some neurodegenerative diseases. For example, the five bundles in FIG. 6A are suspected to be associated with Alzheimer's disease. White-matter bundles open the door to tractometry analysis, in which white-matter metrics (such as the fractional anisotropy, axial diffusivity, radial diffusivity, and mean diffusivity shown in FIG. 3) are analyzed within each bundle. These metrics can be analyzed across the entire bundles, or within sub-regions as shown in FIG. 6B. Note that more advanced metrics based at least in part on high-dimensional local models (be they single- or multi-shell) may also be computed, such as: free-water indices, myelin water fraction, neurite density index, orientation dispersion index, and/or magnetization transfer ratio.

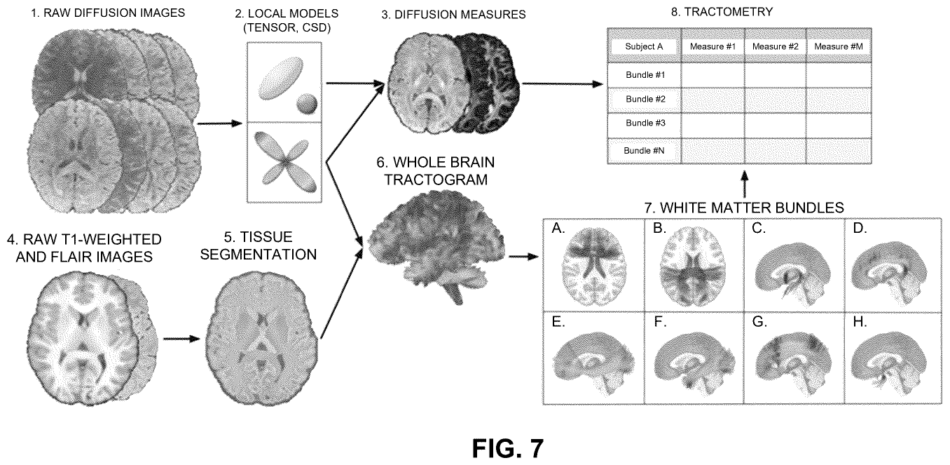

Tractography is a well-established technique for brain connectivity analysis, and has been shown to be effective in measuring the local brain microstructure. While tractography and tractometry can be used independently, they are often used jointly. FIG. 7 presents a drawing of an example of an end-to-end tractometry/tractography pipeline for converting raw input diffusion and structural images into a tractometry report.

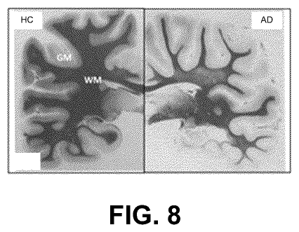

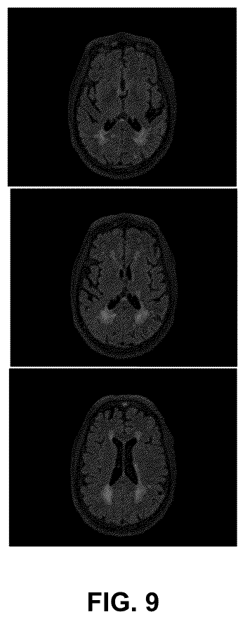

As noted previously, it is often important to study white matter because neurodegenerative diseases can directly impact the structure of the white matter. For example, changes in the white matter with Alzheimer's disease include: microglia activation, loss of oligodendrocytes, demyelination, axonal loss and vascular degeneration. This is shown in FIG. 8, which presents images of an example of a healthy control (HC) and an example of an Alzheimer's disease (AD) brain. Moreover, FIG. 9 presents images of slices of an example of an MRI T₁-weighted image of an Alzheimer's disease patient with dMRI free water hyperintensities overlaid in red. This illustrates that changes with Alzheimer's disease can be localized and measured with dMRI metrics. Similar conclusions have been reached for other diseases, such as multiple sclerosis, Parkinson's disease, and amyotrophic lateral sclerosis. Thus, the scientific community often must look beyond the neuron and the cortex of the medial temporal lobe (where the hippocampus sits) and consider white-matter integrity.

However, in order to assess the integrity of the white matter with diffusion MRI images, typically it is important to confirm that the processing operations that separate raw input diffusion images and tractometry results did not fail to produce meaningful results. This is typically done with so-called quality controls. For example, automatic dMRI QC includes: QC techniques dedicated to finding errors in one specific processing operation (e.g. finding motion artifacts, wrong b-vector distributions, etc.), and QC techniques dedicated to finding problems in the overall processing pipeline. (Note that QC is not to be confused with quality analysis or QA. While QA is the operation of avoiding the occurrence of problems by improving a process, QC is about finding problems in the output of that process.

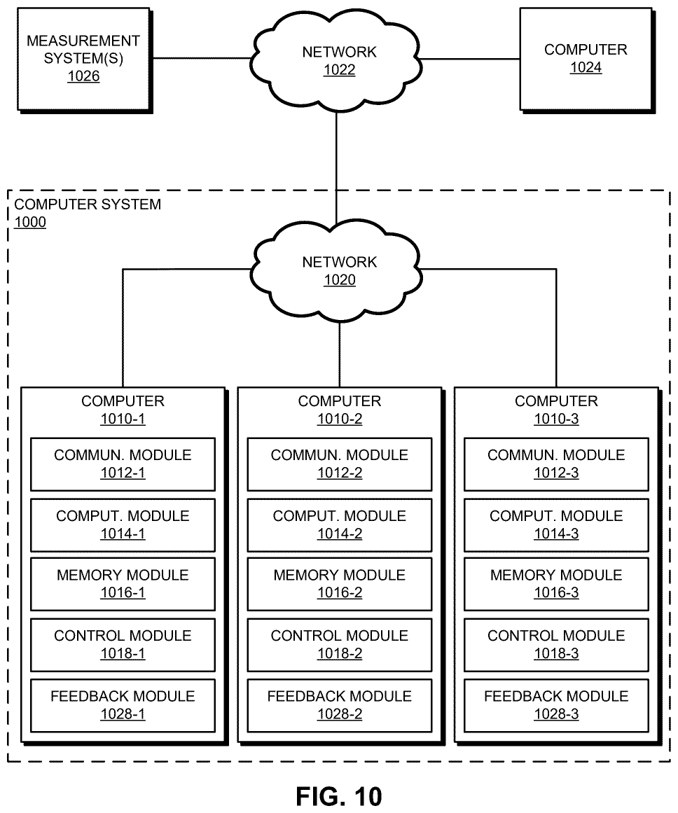

We now describe a computer system that may implement the analysis and QC techniques. FIG. 10 presents a block diagram illustrating an example of a computer system 1000. This computer system may include one or more computers 1010. These computers may include: communication modules 1012, computation modules 1014, memory modules 1016, and optional control modules 1018. Note that a given module or engine may be implemented in hardware and/or in software.

Communication modules 1012 may communicate frames or packets with data or information (such as measurement results or control instructions) between computers 1010 via a network 1020 (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules 1012 may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3rd Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies.

In the described embodiments, processing a packet or a frame in a given one of computers 1010 (such as computer 1010-1) may include: receiving the signals with a packet or the frame; decoding/extracting the packet or the frame from the received signals to acquire the packet or the frame; and processing the packet or the frame to determine information contained in the payload of the packet or the frame. Note that the communication in FIG. 10 may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Note that wireless communication between components in FIG. 10 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. In some embodiments, the communication between the components may use multi-user transmission (such as orthogonal frequency division multiple access or OFDMA).

Moreover, computation modules 1014 may perform calculations using: one or more microprocessors, ASICs, microcontrollers, programmable-logic devices, GPUs and/or one or more digital signal processors (DSPs). Note that a given computation component is sometimes referred to as a ‘computation device’.

Furthermore, memory modules 1016 may access stored data or information in memory that local in computer system 1000 and/or that is remotely located from computer system 1000. Notably, in some embodiments, one or more of memory modules 1016 may access stored measurement results in the local memory, such as dMRI data for one or more individuals (which, for multiple individuals, may include cases and controls or disease and healthy populations). Alternatively or additionally, in other embodiments, one or more memory modules 1016 may access, via one or more of communication modules 1012, stored measurement results in the remote memory in computer 1024, e.g., via network 1020 and network 1022. Note that network 1022 may include: the Internet and/or an intranet. In some embodiments, the measurement results are received from one or more measurement systems 1026 (such as MRI scanners) via network 1020 and network 1022 and one or more of communication modules 1012. Thus, in some embodiments at least some of the measurement results may have been received previously and may be stored in memory, while in other embodiments at least some of the measurement results may be received in real-time from the one or more measurement systems 1026.

While FIG. 10 illustrates computer system 1000 at a particular location, in other embodiments at least a portion of computer system 1000 is implemented at more than one location. Thus, in some embodiments, computer system 1000 is implemented in a centralized manner, while in other embodiments at least a portion of computer system 1000 is implemented in a distributed manner. For example, in some embodiments, the one or more measurement systems 1026 may include local hardware and/or software that performs at least some of the operations in the analysis and QC techniques. This remote processing may reduce the amount of data that is communicated via network 1020 and network 1022. In addition, the remote processing may anonymize the measurement results that are communicated to and analyzed by computer system 1000. This capability may help ensure computer system 1000 is compatible and compliant with regulations, such as the Health Insurance Portability and Accountability Act, e.g., by removing or obfuscating protected health information in the measurement results.

Although we describe the computation environment shown in FIG. 10 as an example, in alternative embodiments, different numbers or types of components may be present in computer system 1000. For example, some embodiments may include more or fewer components, a different component, and/or components may be combined into a single component, and/or a single component may be divided into two or more components.

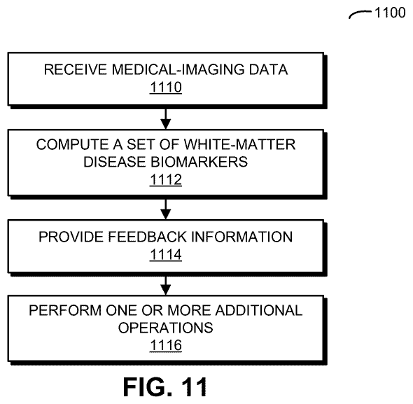

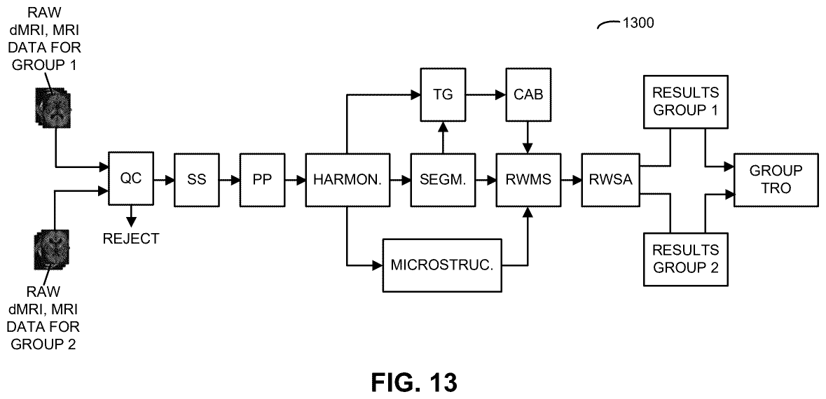

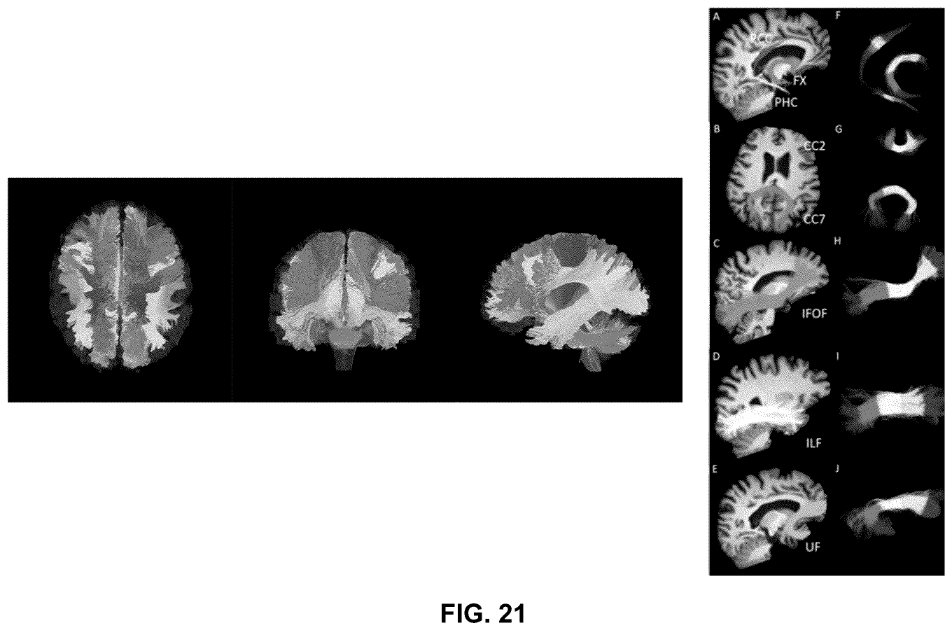

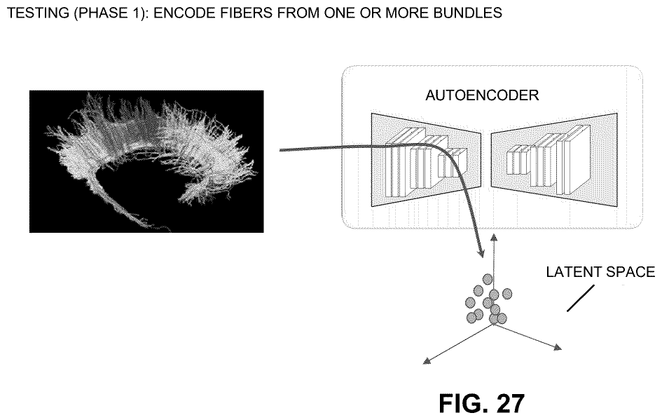

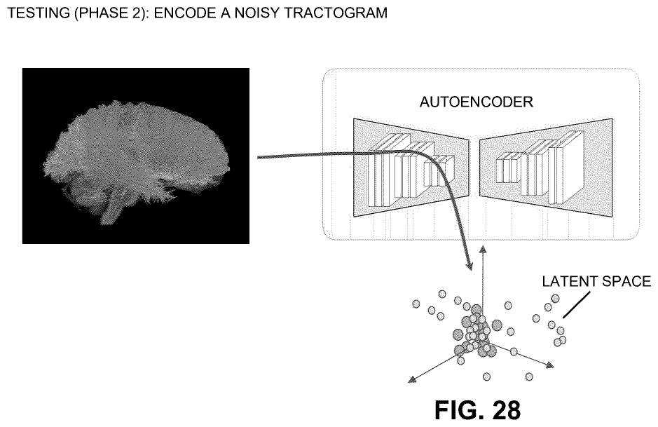

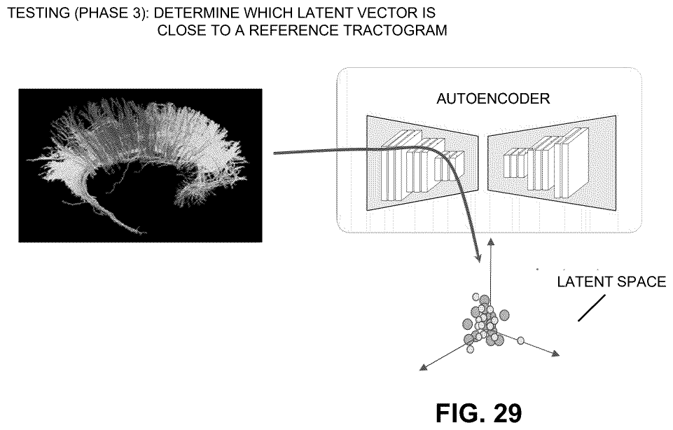

As discussed previously, existing analysis techniques often suffer from a number of problems. Moreover, as described further below with reference to FIGS. 11-33, in order to address these challenges computer system 1000 may perform the analysis techniques. Notably, during the analysis techniques, one or more of optional control modules 1018 may divide the analysis among computers 1010. Then, a given computer (such as computer 1010-1) may perform at least a designated portion of the analysis. In particular, computation module 1014-1 may receive (e.g., access) information (e.g., using memory module 1016-1) specifying medical-imaging data that specify the central nervous system (including white matter) for one or more individuals. Note that the medical-imaging data may include or may correspond to dMRI data. Then, computation module 1014-1 may perform operations in multiple stages in an analysis pipeline. For example, as described further below with reference to FIGS. 13-33, the analysis pipeline may include: QC (such as visual inspection, an image resolution check and/or a gradient distribution check), skull stripping or SS, preprocessing or PP (such as denoising, motion correction and/or a correction for magnetic field inhomogeneity), harmonization (to correct for data variation, e.g., in dMRI and/or MRI data, associated with different MR scanners), tissue segmentation (e.g., white matter, grey matter, tumor, cerebrospinal fluid, etc., and which may be based at least in part on structural MRI data using a convolutional neural network). The output of these portions or stages of the analysis pipeline may include tractography results (which are sometimes referred to as ‘tractograms’) that specify a set of neurological fibers, which are sometimes referred to as ‘neural tracts’ or ‘streamlines’. In the present discussion, note that a ‘neurological fiber’ includes a sequence of three dimensional or 3D points that are connected together.

Moreover, computation module 1014-1 may perform additional tractography by computing, using a predetermined (e.g., pretrained) autoencoder neural network, second tractography results that specify a second set of neurological fibers based at least in part on the tractography results and information associated with a neurological anatomical region. Note that a subset of the set of neurological fibers may be anatomically implausible and the second set of fibers may exclude the subset. Moreover, the predetermined autoencoder neural network may be trained using an unsupervised-learning technique. In some embodiments, the second set of neurological fibers may, at least in part, be different from the set of neurological fibers. Note that computing the second tractography results may include a cleaning and bundling operation in which the neurological fibers are grouped or bundled into different types of bundles of neurological fibers having different numbers of neurological fibers.

Next, computation module 1014-1 may perform additional operations in multiple stages in the analysis pipeline. For example, the second tractography results may be input to or used by one or more stages or operations in the analysis pipeline, such as: statistical analysis, connectome analysis, population analysis, etc. Notably, the analysis pipeline may include: microstructure analysis, region-wise microstructure statistics (RWMS), and/or region-wise statistical analysis (RWSA). In the case of analysis for an individual, region-wise statistical analysis may be based at least in part on a comparison with a reference atlas or data structure (such as a region-wise microstructure brain atlas corresponding to multiple individuals). Alternatively, in the case of analysis of a population, at least a portion of the analysis may be computed based at least in part on cases and controls in the population.

After performing the operations in the stages in the analysis pipeline, computation module 1014-1 may output a subset of white-matter disease biomarkers for different neurological anatomical regions for at least the designated portion of the analysis. For a given neurological anatomical region, the set of white-matter disease biomarkers may include: an apparent fiber density, which corresponds to a total intra-axonal volume; an amount of free water; or a demyelination metric. Note that the amount of free water may correspond to neuroinflammation, and/or the apparent fiber density may correspond to axonal disruption or axonal quality. The apparent fiber density may be recovered or determined using: a diffusion fractional anisotropy, water corrected fractional anisotropy, water corrected axial diffusivity, intracellular neurite orientation dispersion and density imaging (NODDI) volume fraction, and/or a local fODF. Moreover, the demyelination metric may include: an inverse free-water-corrected radial diffusivity, a ratio of T₁ to T₂, a magnetization transfer ratio, or MWF. As noted previously, the MWF may be the ratio of the signal amplitudes measured in or associated with two water compartments

$$MWF = \frac{MW}{( MW + EICW )} ,$$

where MW is the signal amplitude associated with a myelin water compartment and EICW is a signal amplitude associated with an extra-intra cellular water compartment. These signal amplitude values may be obtained via a multi-exponential function and nonlinear optimization from a series of acquired T₂-weighted images at different echo times (e.g., up to 48 echoes). In some embodiments, the set of white-matter disease biomarkers may be computed on a per-voxel basis and/or a per-neurological-fiber (or fixel) basis.

Furthermore, as described further below with reference to FIGS. 8 and 34-42, one or more feedback modules 1028 in computer system 1000 may use the set of white-matter disease biomarkers for different neurological anatomical regions to provide feedback information associated with at least the individual or the population. For example, the set of white-matter disease biomarkers for different neurological anatomical regions may be inputs to a pretrained predictive model (such as a supervised machine-learning model or a neural network), which outputs the feedback information. Note that the feedback information may include: diagnostic information, information associated with disease progression (such as a disease stage), information regarding efficacy of a treatment, or a treatment recommendation (e.g., based at least in part on the disease stage). In some embodiments, the feedback information (such as the diagnostic information) may be based at least in part on a volume of a neurological anatomical region in at least the individual, such as the hippocampus.

In these ways, computer system 1000 may automatically and accurately analyze white matter for at least an individual or a population, such as: perform filtering and/or grouping or bundling of the neurological fibers, computing the set of white-matter disease biomarkers, etc. These capabilities may allow computer system 1000 to perform subsequent analyses or one or more additional operations, such as: connectome analysis, white matter segmentation, one or more clinical trial enrollment or exclusion criteria, assessing the impact of a medical intervention for a disease (e.g., in a clinical trial for a candidate pharmaceutical agent, neurostimulation and/or another type of therapy), precision medicine (such as in selecting a correct medical intervention to treat a disease, e.g., as a companion diagnostic for a prescription drug or a dose of a prescription drug), etc. For example, the more-accurate white-matter results may allow more accurate determination of: an axon density index, radial diffusion, volume, a myelin index or metric, an inflammation index or metric, fractional anisotropy (which is sensitive to anomalies), free water, apparent fiber density, a microglia index or metric, an astrocyte index or metric, an oligodendrocyte index or metric, a lesion edema index or metric, a cerebrospinal fluid index or metric and/or, more generally, a characteristic or attribute associated with microstructure environment (e.g., on a sub-millimeter length scale that is less than a length scale corresponding to a voxel) of one or more neurological fibers (and, more generally, white matter) in particular neurological anatomic regions. Note that the disease may include: Parkinson's disease, Alzheimer's disease, multiple sclerosis, amyotrophic lateral sclerosis (ALS) or Lou Gehrig's disease, normal pressure hydrocephalus, concussion, migraine, epilepsy, a type of cancer, an autoimmune disease, schizophrenia, depression, bipolar disorder, another type of mental illness, traumatic brain injury, an alcohol-use disorder, a neurodegenerative disease, a neuroinflammatory disease, or another type of neurological disease of the central nervous system. Consequently, the analysis techniques may facilitate accurate, value-added use of the measurement results, such as medical-imaging data.

We now describe embodiments of the method. FIG. 11 presents a flow diagram illustrating an example of a method 1100 for providing feedback information, which may be performed by a computer system (such as computer system 1000 in FIG. 10). During operation, the computer system may receive medical-imaging data (operation 1110) associated with at least an individual or one or more individuals. For example, the receiving may include accessing the information in memory. Note that the medical-imaging data may be associated with magnetic-resonance images of at least the individual or the one or more individuals, including: T₁-weighted, T₂-weighted, proton density, fluid-attenuated inversion recovery (FLAIR), b-value images, diffusion weighted images, diffusion tensor images, high angular resolution diffusion images, and/or another type of magnetic-resonance image or data. Thus, the medical-imaging data may include dMRI data.

Then, the computer system may compute, based at least in part on the medical-imaging data, a set of white-matter disease biomarkers (operation 1112) for different neurological anatomical regions, where, for a given neurological anatomical region, the set of white-matter disease biomarkers includes an apparent fiber density, which corresponds to a total intra-axonal volume. In the present disclosure, for a voxel v and neurological fiber bundle b that goes through v, the apparent fiber density of v is the apparent proportion of its volume that is occupied by the neurological fibers of b (‘apparent’ because we cannot actually count the neurological fibers because they are too small). An apparent fiber density of, e.g., 0 means that no neurological fiber of b crosses that the voxel v and a value of, e.g., 1 means that v is fully occupied by the neurological fibers of b. The apparent fiber density of v may be recovered or determined using a diffusion fractional anisotropy, water corrected fractional anisotropy, water corrected axial diffusivity, intracellular NODDI volume fraction and/or a local fiber orientation distribution function. It may also be computed by considering the amplitude of the neurological fiber ODF lobe aligned with b. Note that each voxel v overlapped by more than one neurological bundle may be assigned several apparent fiber density values, one for each neurological bundle.

Moreover, for the given neurological anatomical region, the set of white-matter disease biomarkers may include: an amount of free water or a demyelination metric. Note that the amount of free water may correspond to neuroinflammation, and/or the apparent fiber density may correspond to axonal disruption or axonal quality. Furthermore, the demyelination metric may include or may correspond to: an inverse free-water-corrected radial diffusivity, a ratio of T₁ to T₂, and/or a magnetization transfer ratio. Additionally, the set of white-matter disease biomarkers may be computed on a per-voxel basis and/or a per-neurological-fiber or fixel basis.

In the present disclosure, the amount of free water or the free-water index approximates the proportion of water contained in a voxel v of the brain. Free water may be determined from a corrected diffusion tensor imaging isotropic compartment, the isotropic fraction of NODDI and/or the isotropic compartment of any multi-compartmental diffusion MRI model. For example, we may approximate the local diffusion signal in v by the weighted sum of two tensors: a water tensor (WT) that accounts for the liquid in v, and a structural tensor or ST (which is sometimes referred to as a ‘tissue tensor’) that accounts for the non-liquid water-tensor tissue in v. Consequently, for v:

$${dMRI}_{v} = α \cdot {W}_{v} + ( 1 - α ) \cdot {ST}_{v} ,$$

where alpha is a value between, e.g., 0 and 1 which, in turn, is the free-water index.

Moreover, in the present disclosure, the demyelination metric or the myelin index (MI) measures the apparent amount of myelin contained in a voxel v. As noted previously, depending on the MRI acquisition protocol and the available modalities, the myelin index may be approximated by several measures such as the free-water corrected radial diffusivity, the T₁/T₂ ratio, the radial complement of the apparent fiber density, the magnetization transfer ratio or the myelin water fraction.

Next, the computer may provide the feedback information (operation 1114) associated with at least the individual based at least in part on the computed set of white-matter disease biomarkers in different neurological anatomical regions. Moreover, the feedback information may include: diagnostic information, information associated with disease progression (such as a disease stage), information regarding efficacy of a treatment, or a treatment recommendation (e.g., based at least in part on the disease stage). For example, the diagnostic information may be associated with a neurological or neurodegenerative disease, including: epilepsy, depression, autism, cerebral palsy, multiple sclerosis, Alzheimer's disease, Parkinson's disease, amyotrophic lateral sclerosis, traumatic brain injury, an alcohol-use disorder, or another neurological or neurodegenerative disease. Furthermore, the feedback information may be based at least in part on a volume of a neurological anatomical region in at least the individual, such as the hippocampus.

In some embodiments, the computer system may optionally perform one or more additional operations (operation 1116). For example, the medical-imaging data may be associated with a population of cases and controls, which includes at least the individual, and the feedback information may be associated with the population.

Moreover, the feedback information is determined using a pretrained predictive model. For example, the pretrained predictive model may include: a machine-learning model or a neural network. Note that a given node in a given layer in the neural network may include an activation function, and the activation function may include: a ReLU, a leaky ReLU, an ELU activation function, a parametric ReLU, a tanh activation function, or a sigmoid activation function.

Furthermore, the computer system may dynamically determine the neurological anatomical regions that are of interest based at least in part on changes (e.g., as a function of time relative to previously computed values for at least the individual) of the set of white-matter disease biomarkers and/or based at least in part on a type of disease (such as predetermined pathophysiology associated with a type of disease). Additionally, the feedback information may be based at least in part on the dynamically determined neurological anatomical regions.

In some embodiments of method 1100, there may be additional or fewer operations. Furthermore, the order of the operations may be changed, and/or two or more operations may be combined into a single operation.

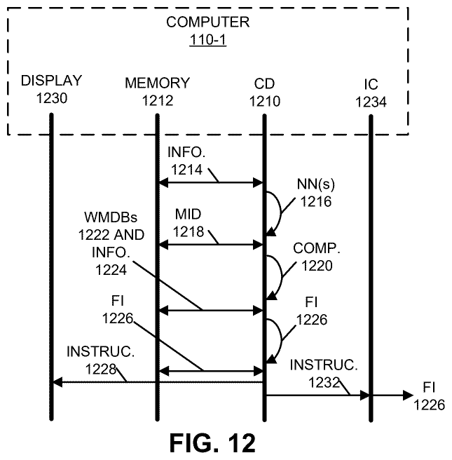

Embodiments of the analysis techniques are further illustrated in FIG. 12, which presents a drawing illustrating an example of communication among components in computer system 1000. In FIG. 12, a computation device (CD) 1210 (such as a processor or a GPU) in computer 1010-1 may access, in memory 1212 in computer 1010-1, information 1214 specifying configuration instructions and hyperparameters for one or more predetermined or pretrained models, such as one or more neural networks (NNs) 1216. After receiving the configuration instructions and the hyperparameters, computation device 1210 may implement the one or more neural networks 1216.

Moreover, computation device 1210 may access in memory 1212 information specifying medical-imaging data 1218 that specify white matter for at least an individual. After receiving medical-imaging data 1218, computation device 1210 may compute 1220, using the one or more neural networks 1216 and based at least in part on the medical-imaging data 1218, a set of white-matter disease biomarkers (WMDBs) 1222 for different neurological anatomical regions (as specified by information 1224), where, for a given neurological anatomical region, the set of white-matter disease biomarkers includes: an apparent fiber density, which corresponds to a total intra-axonal volume; an amount of free water; and/or a demyelination metric. After or while performing the computations, computation device 1210 may store results, including the set of white-matter disease biomarkers 1222 and information 1224, in memory 1212.

Next, computation device 1210 may determine feedback information (FI) 1226 associated with at least the individual based at least in part on the computed set of white-matter disease biomarkers 1222 in different neurological anatomical regions. This feedback information may be stored in memory 1212. Alternatively or additionally, computation device 1210 may provide instructions 1228 to a display 1230 in computer 1010-1 to display feedback information 1226. In some embodiments, computation device 1210 may provide instructions 1232 to an interface circuit 1234 in computer 1010-1 to provide feedback information 1226 to another computer or electronic device (not shown).

While FIG. 12 illustrates communication between components using unidirectional or bidirectional communication with lines having single arrows or double arrows, in general the communication in a given operation in this figure may involve unidirectional or bidirectional communication.

In some embodiments, the set of white-matter disease biomarkers includes free-water, apparent-fiber-density and myelin metrics. These white matter disease biomarkers may be used in conjunction with different neurological diseases or neurodegenerative diseases. For example, the neurological diseases may include: Alzheimer's disease, Parkinson's disease, multiple sclerosis, traumatic brain injury, depression, amyotrophic lateral sclerosis and/or chronic traumatic encephalopathy.

In the discussion that follows, Alzheimer's disease, Parkinson's disease and multiple sclerosis are used as illustrative examples. Because Alzheimer's disease, Parkinson's disease and multiple sclerosis are, in general, slowly progressing neurological diseases, they often have corresponding signatures associated with them. Notably, in their early stages, Alzheimer's disease, Parkinson's disease and multiple sclerosis are characterized by inflammation of the white matter, which typically causes the free-water metric to increase. At this stage, the diseases are often unknown to the patients and the apparent-fiber-density and myelin metrics are usually stable. However, in later disease stages, the apparent-fiber-density and myelin metrics often start to decrease, thereby showing signs of axonal loss and myelin degradation. Consequently, Alzheimer's disease, Parkinson's disease and multiple sclerosis are typically characterized by an increase of free water and, in later stages of these diseases, by a decrease of the apparent-fiber-density and myelin metrics.

During clinical trials, it is important to know if a particular medication is effective or not. Interestingly, when a medication is effective, the free water typically decreases (which indicates that there is less inflammation in the white matter). In some cases, an increase of the apparent-fiber-density and myelin metrics are also observed, which suggests that the medication has the effect of remyelinizing the white matter and rebuilding axons. It is hypothesized that this happens when patients are in the ‘middle stage’ of their diseases, where myelin and axons have not been irreversibly damaged.

For traumatic brain injury, immediately following brain concussion, an increase of free water is observed because of sudden neuro-inflammation and swelling. In most cases, the free water decreases after some time as the symptoms disappear. In these cases, the apparent-fiber-density and myelin metrics are stable all along. However, in some cases, the traumatic brain injury neuroinflammation becomes chronic which in turn can affect the integrity of the white matter and lead to reduction in apparent fiber density and myelin metrics.

Thus, for Alzheimer's disease, Parkinson's disease, multiple sclerosis and traumatic brain injury, there is an increase of free water followed in time by a decrease of the apparent-fiber-density and myelin metrics.

Note that where these changes occur in the brain may depend on the neurological diseases. For example, for Alzheimer's disease the changes may occur in the fornix region and in ‘Alzheimer's disease bundles.’ Alternatively, for Parkinson's disease the changes may occur in the substantia nigra and ‘Parkinson's disease bundles.’ Moreover, for multiple sclerosis the changes may occur in the vicinity of white-matter lesions and ‘multiple sclerosis bundles.’ Furthermore, for traumatic brain injury the changes may occur in a variety of locations in the brain, but there are some ‘traumatic brain injury bundles.’ Additionally, note that Alzheimer's disease, Parkinson's disease, and multiple sclerosis are often associated with an atrophy of the hippocampal volume.

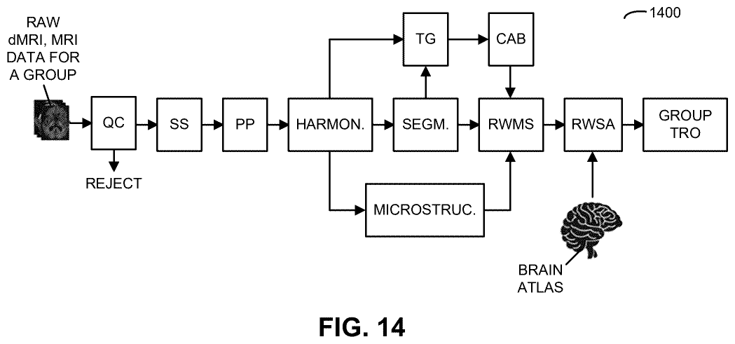

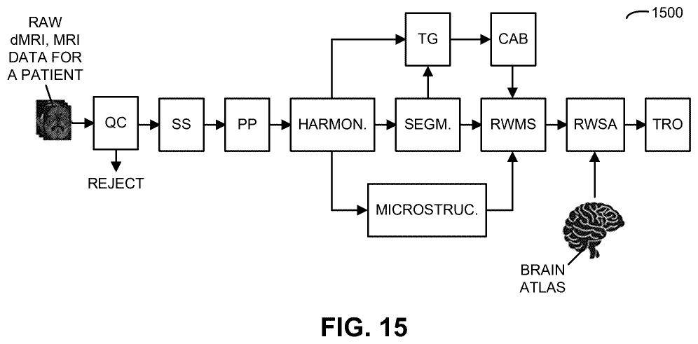

We now further describe embodiments of an analysis pipeline. FIG. 13 presents a drawing illustrating an example of an analysis pipeline 1300 for use with a population, including N cases and M controls (where N and M are non-zero integers). FIG. 14 presents a drawing illustrating an example of an analysis pipeline 1400 for use with a population of multiple individuals, in which comparisons are made relative to a region-wise microstructure brain atlas. FIG. 15 presents a drawing illustrating an example of an analysis pipeline 1500 for use with at least an individual, in which comparisons are made relative to a region-wise microstructure brain atlas.

As discussed previously, in some embodiments the analysis pipeline may include: QC (such as visual inspection, an image resolution check and/or a gradient distribution assessment), skull stripping or SS, preprocessing or PP (such as denoising, motion correction and/or a correction for magnetic field inhomogeneity), harmonization or HARMON (to correct for data variation, e.g., in dMRI and/or MRI data, associated with different MR scanners), tissue segmentation (e.g., white matter, grey matter, tumor, cerebrospinal fluid, etc., and which may be based at least in part on structural MRI data using a convolutional neural network), tractography or TG (which may be followed by a clean and bundle or CAB operation using an autoencoder neural network), microstructure analysis (which may determine fractional anisotropy, mean diffusivity, free water, etc.), region-wise microstructure statistics (RWMS), region-wise statistical analysis (RWSA), and/or a diagnostic or treatment recommendation operation or TRO (and, more generally, a feedback operation) for a group or population, or for an individual. In the case of analysis for an individual in FIG. 15, region-wise statistical analysis may be based at least in part on a comparison with a reference atlas or data structure (such as the region-wise microstructure brain atlas corresponding to multiple healthy or normal individuals, e.g., 10,000-50,000 individuals, which may be separated by gender, age, right or left handedness, etc.). Alternatively, in the case of analysis of a population, the decision or treatment recommendation operation may be computed based at least in part on cases and controls in the population (FIG. 13), or based at least in part on a comparison with a reference atlas or data structure (FIG. 14).

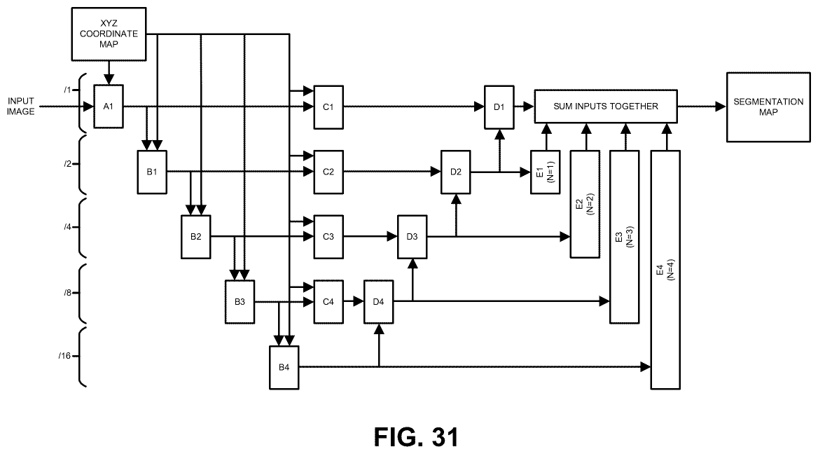

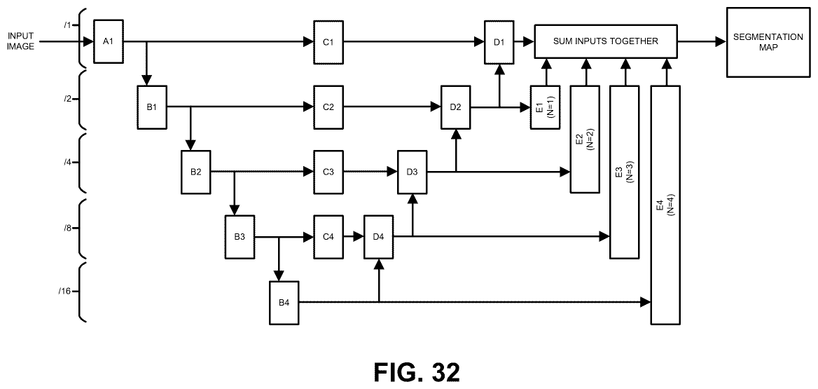

Note that at least some of the operations in embodiments of the pipeline are performed using pretrained models, such as machine learning models and/or neural networks. For example, at least a portion of the QC, the skull stripping, the preprocessing, the harmonization, the tissue segmentation, the tractography and/or the diagnostic or treatment recommendation operation may be performed by corresponding pretrained models, while other operations (such as the microstructure analysis, the region-wise microstructure statistics and/or the region-wise statistical analysis) may be performed using non-machine-learning techniques (such as statistical analysis). Illustrations of architectures for several pretrained neural networks are described further with reference to FIGS. 31-32. Note that at least some of the neural networks may be trained using labeled real images. However, in other embodiments, at least some of the neural networks may be training using synthetic or artificially generated images.

In some embodiments the analysis pipeline may be used to analyze a large amount of medical-imaging data that may have been acquired using different MRI scanners (e.g., during analysis of a population), different acquisition protocols (such as different pulse sequences) and/or at different times. For example, at least some of the medical-imaging data may be acquired using: different radio-frequency coils, different pulse sequences, different magnetic field strengths (such as 1.5 or 3 T), etc. Moreover, the reconstruction techniques that are used to convert 4D measurements into real-space medical images make cause the medical images to be non-uniform or non-standardized. Consequently, there may be differences in the medical-imaging data that is input to the analysis pipeline on a per-patient basis.

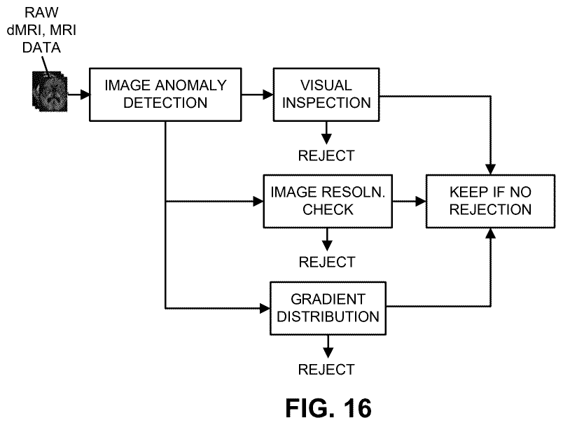

Therefore, as discussed previously, the analysis pipeline may include QC to identify and/or reject medical images with artifacts (such as missing images, frame or slide drops, blurring due to motion of a given individual during the measurements, magnetic artifacts, etc.). This is shown in FIG. 16, which presents a drawing illustrating an example of QC in an analysis pipeline. These embodiments of QC may include: an image anomaly detection followed by visual inspection; an image resolution check; and a gradient distribution assessment. The image anomaly detection may include: slice drop, magnetic artifacts, blurring, etc. Moreover, the image anomaly detection may produce a report that may be used during an optional visual inspection. Alternatively, in some embodiments the visual inspection may be automatically performed, e.g., using a pretrained model (such as a machine-learning model or a neural network). Images that include anomalies may be rejected.

Furthermore, the image resolution check may determine whether the image (voxel) resolution is, e.g., 1 mm³ or 2 mm². Images with different resolutions may be rejected.

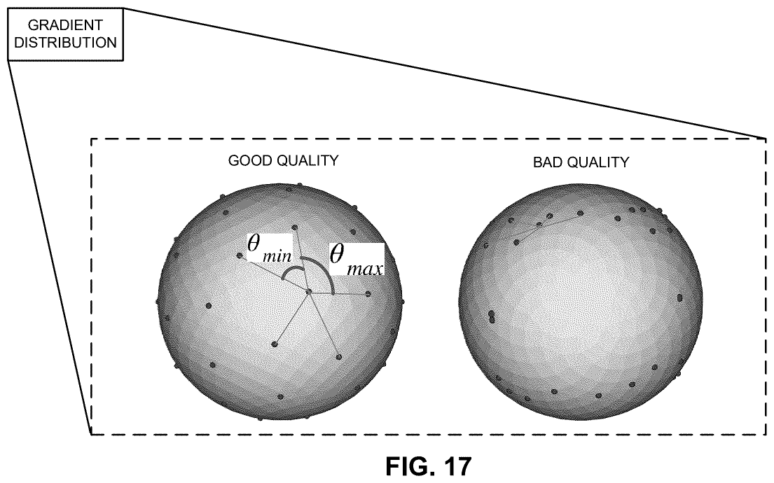

Additionally, the gradient distribution assessment may determine an angular distribution of orientation, e.g., on a sphere, which may be indicative of how freely water moves in an image. This is shown in FIG. 17, which presents a drawing illustrating an example of determination of good versus bad quality using a gradient distribution assessment during the QC in FIG. 16. Notably, good quality may be associated with an approximately uniform angular distribution with approximately equal minimum and maximum angles, while bad quality may be associated with a narrower angular distribution with different minimum and maximum angles. Images with orientations that are nonuniform (such as with absolute differences between the minimum and maximum angle above 20°/M degrees, where M is the non-zero integer number of neighbors that are taken into consideration (e.g., M equals 5 in FIG. 17). In some embodiments, an angular configuration may be rejected when the local density of M nearest neighbors is twice as large as the proportion of the convex area they occupy on the sphere.

During skull stripping, the medical images are segmented. Notably, voxels that do not correspond to or include brain tissue (such as the eyes, nose, mouth and skin) may be forced to zero. This operation may be performed using a pretrained neural network (as illustrated further below with reference to FIG. 31), which is trained using images for, e.g., 2000 individuals.

Moreover, during preprocessing, the medical images may be denoised, e.g., using a principal component technique. The preprocessing may also correct for motion of an individual during the measurement of the medical images (which can take up to an hour). For example, temporally adjacent or proximate medical images may be compared and may be regularized relative to each other. Furthermore, the preprocessing may correct for magnetic field inhomogeneity in a given MRI scanner. These magnetic field inhomogeneities may include low-frequency intensity variations present in MRI data (which are sometimes referred to as a ‘bias field’) that can be compensated for with a non-parametric technique that estimates the bias field and removes it from the source image (e.g. an N3 and/or an N4 bias-field correction technique). Preprocessing may also correct susceptibility artifacts. Notably, when two adjacent or proximate materials (such as bone and air) have different susceptibility, the magnetic field will have an inhomogeneous distribution that results in a local warping/distortion of the image. In some embodiments, this may be addressed using two or more acquisitions with different parameters, so that the structural content of the images may be the same but the distortions are different. From these images, an inverse transformation may be computed to compensate for these distortions. This may be performed using analysis tools, such as Topup (from the Oxford Centre for Functional MRI of the Brain, Oxford, United Kingdom) or Eddy (from the Oxford Centre for Functional MRI of the Brain, Oxford, United Kingdom).

Additionally, during harmonization, data variation in the medical images associated with different MR scanners may be performed. This may be important in embodiments where the medical images include aggregated data. Note that the harmonization may include separate operations for MRI data and dMRI data. During harmonization of MRI data, the image intensity of the images may be standardized. For example, the greyscale (0 to 1) of a given image may be globally adjusted so that the images have the same average value.

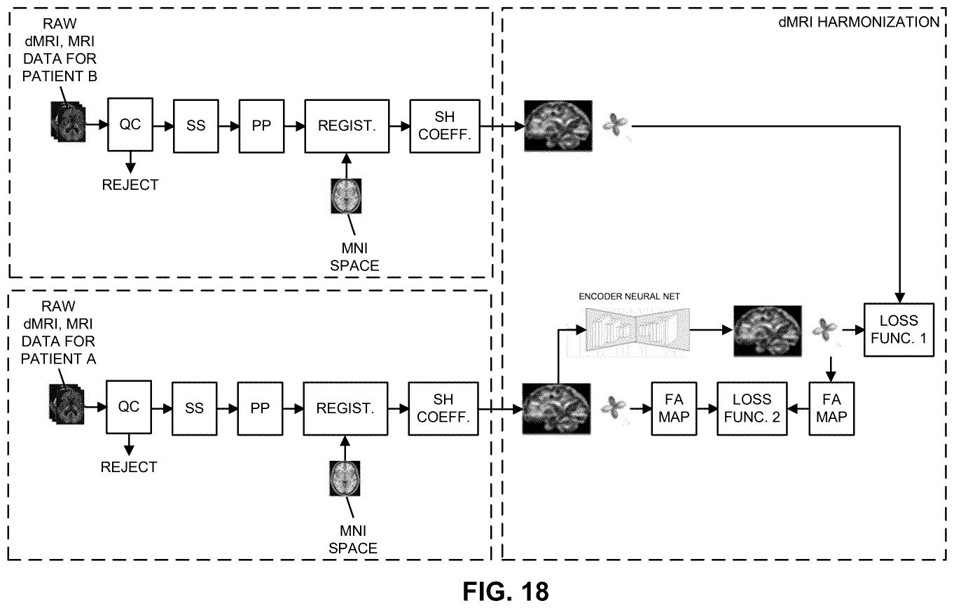

FIG. 18 presents a drawing illustrating an example of self-supervised training of dMRI harmonization in an analysis pipeline. Notably, after QC, skull stripping and preprocessing, the dMRI data may be registered, e.g., using a standard brain defined by the MNI, which is sometimes referred to as an ‘MNI space.’ Then, spherical harmonic (SH) coefficients are determined, which summarize the signal in the dMRI data. This produces a map of glyphs of diffusion signal in the brain. The encoder/decoder neural network produces a modified homogeneous map of glyphs, which is compared to the results from another patient in loss function 1. Moreover, fraction anisotropy maps, which measure how elongated a glyph is on a per-voxel basis, are compared by loss function 2.

Note that self-supervised learning stands for learning techniques that use the raw data (and not a reference annotation) to train a model. In the embodiments illustrated in FIG. 18, the brain of a patient A is used as a target for patient B. Patient A and B can be the same person whose MRIs were acquired following different protocols or different persons sharing similar characteristics (both healthy, same age, same handedness, same gender, same education level, etc.). The loss functions 1 and 2 in FIG. 18 may be an arbitrary regression loss function, such as: L1 or L2 distance, a Huber loss, a Tukey loss, etc.

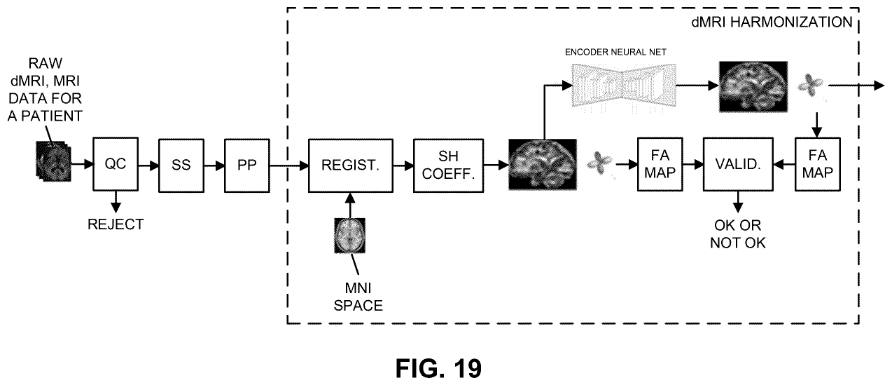

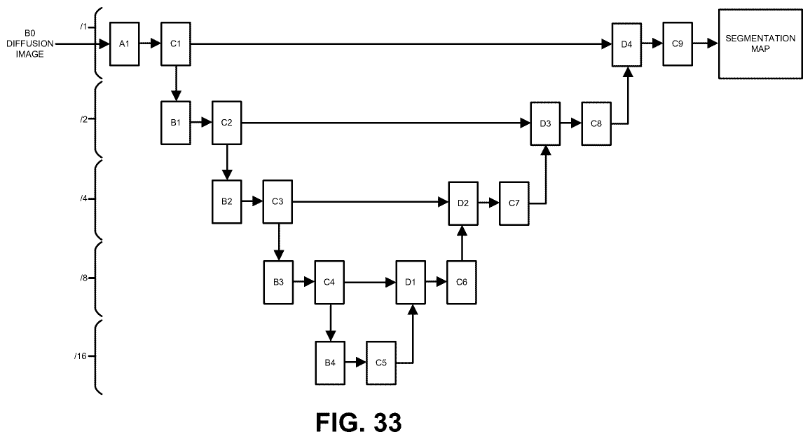

After training, the resulting encoding/decoding neural network may be used to harmonize dMRI images (with reference to a neural network, such as that described further below with reference to FIG. 33, but with dMRI data at its input and output). FIG. 19 presents a drawing illustrating an example of dMRI harmonization in an analysis pipeline. Notably, the fraction anisotropy maps of the glyphs with and without processing using the encoder/decoder neural network are compared in a validation operation to determine whether a particular image is acceptable. The harmonization may be performed by a pretrained neural network that was trained to harmonize data. In some embodiments, acceptable criteria may include: an average difference of more than 10% in the overall white matter, and/or a local difference of more than 20%.

While harmonization can be applied to the diffusion signal (as illustrated in FIGS. 15, 18 and 19), in some embodiments it may also be applied to feature maps (such as a free-water or FW map, an apparent fiber density or AFD map, a myelin map, etc.). These feature map harmonization(s) may be deep-learning based (e.g., using a pretrained neural network) or non-deep learning base (e.g., by adjusting the image histogram to a reference histogram or using a technique such as ComBAT from the University of Pennsylvania of Philadelphia, Pennsylvania).

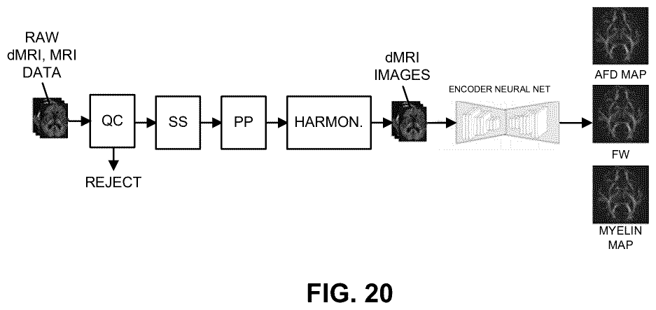

In some embodiments, the analysis pipeline may use a pretrained neural network to recover white-matter metrics (such as apparent fiber density, free water and/or a myelin index) from low-resolution dMRI data. Stated differently, this pretrained neural network may recover the white-matter metrics when we do not have the right data (e.g., sufficient data) to compute the white-matter metrics with one of the aforementioned approaches. This is shown in FIG. 20, which presents a drawing illustrating an example of conversion of low-resolution dMRI images (which include information about the ADC) into white-matter maps in an analysis pipeline. Notably, the white-matter maps may include: the apparent-fiber-density map, the free-water map, and/or the myelin map.

During tissue segmentation, white matter, grey matter, tumor, cerebrospinal fluid, a potential tumor and/or smaller regions in the brain may be segmented based at least in part on structural MRI data using a convolutional neural network. This operation may be performed using a pretrained neural network that uses a 3D convolution to generate a segmentation map (as illustrated below with reference to FIG. 32). Note that the segmentation map may be used in the tractography operation of the analysis pipeline. In some embodiments, the tissue segmentation is performed using: a U-Net neural network architecture or configuration (from the Computer Science Department at the University of Freiburg, Freiburg im Breisgau, Germany), V-Net neural network architecture or configuration (from the Computer Science Department at the University of Freiburg, Freiburg im Breisgau, Germany), E-Net (from the Faculty of Mathematics, University of Perdue, Perdue, Indiana, and/or DeepLab (from Google, Inc., Mountain View, California).

Tractography uses local orientation information from dMRI data to delineate brain white matter fiber pathways, and thus, to provide information about its structural connectivity. These connectivity pathways in tractograms are composed of streamlines virtually representing fascicles of white matter fibers. Note that the quality of the tractograms influence various aspects of connectome analysis, including: the connectivity density, the clustering degree or the existence of connections themselves, among others.

Existing tractography techniques typically face a number of challenges when propagating streamlines. For example, existing tractography techniques often have difficulty: avoiding the early termination of the tracking procedure; providing streamlines between gray matter regions that are known to be connected while avoiding spurious streamlines; ensuring the full occupancy of the white matter volume by the streamlines; and providing a complete gray matter surface coverage when streamlines reach the cortex.

Consequently, and despite the ongoing efforts to address these issues, white matter tracking techniques are often known to produce a disproportionately large number of invalid streamlines (which are sometimes referred to as ‘implausible streamlines’). The category of invalid streamlines spans a broad group of streamlines that violate accepted neuroanatomical constraints attributed to fiber populations. These include streamlines that contain loops or sharp bends; streamlines that stop in non-gray matter tissues, such as the cerebrospinal fluid; streamlines that prematurely stop in the white matter; or streamlines describing trajectories between gray matter regions that are not connected structurally. Indeed, recent work indicates that, as a trade-off between sensitivity and specificity, existing tractography techniques produce a non-negligible proportion of invalid or non-existing, false-positive streamlines or connections.

To address these problems, many existing tractography techniques use filtering to detect and remove anatomically implausible streamlines, and to mitigate some of the limitations derived from current streamline propagation techniques. Notably, existing diffusion MRI tractography techniques generate tractograms that may contain several million candidate streamlines representing white matter fiber populations. However, a regular tractogram including only anatomically plausible streamlines can contain in the order of 500,000 to 3,000,000 streamlines. Automatic tractography filtering of the implausible streamlines is currently based at least in part on one or more factors, including: streamline geometry features; region-of-interest-driven streamline inclusion and exclusion; clustering (which is sometimes referred to as ‘bundling’); connectivity-based; and/or diffusion signal mapping or forward models.

These existing filtering approaches typically involve one or more assumptions, such as an assumed constraint. For example, invalid streamlines may be identified and removed based at least in part on: an unfeasible streamline length (and, more generally, neuroanatomical constraints); local curvature indices; streamline inclusion and exclusion criteria; and/or white matter and/or tissue local and connectivity constraints for streamline traversal. Alternatively, in clustering approaches, a similarity measure (e.g., based at least in part on assumed defined distance measure) is used to remove non-meaningful data. Moreover, in connectivity-based approaches, undesired streamlines are usually removed by imposing a regularization constraint on the tractogram-derived connectivity matrices. Furthermore, forward models often identify a subset of streamlines to be preserved in a whole-brain tractogram by, e.g., modifying a local fODF based at least in part on the diffusion signal or using local models to weigh the contribution of the diffusion signal to the streamline representation.

In principle, streamline filtering can be seen as an application of choice for deep learning classification techniques. However, neural networks are usually not easily applied to brain tractography, e.g., because of the difficulty in building a labeled dataset for use in supervised training. Indeed, finding irreproachable ground truth streamlines is typically a very difficult endeavor, with significant differences in reproducibility measures even among well-trained expert neuroanatomists. Moreover, depending on the task at hand, the labeling of the streamlines often vary considerably, and thus may require time-consuming manual verification and/or relabeling. Additionally, because existing neural network approaches are trained using supervised-learning techniques, the resulting neural networks either have a fixed number of target classes (such as a fixed number of cluster types or bundles of anatomically coherent streamlines) or use distinctly trained neural networks or models for corresponding types of clusters. Hence, regular classification neural networks are trained to predict a fixed number of classes, and thus cannot be used to predict a different set of classes without being retrained on a newly labeled set of data. These limitations constrain the flexibility of the analysis, and result in additional complexity and computational overhead, or suffer from trade-offs between accuracy and computational efficiency based at least in part on the bundle granularity.

In the analysis techniques, these problems can be addressed by using a deep autoencoder in the embodiments of the analysis pipeline for streamline-based tractography filtering (which is sometimes referred to as ‘filtering in tractography using autoencoders’ or FINTA). In FINTA, the autoencoder may be trained on non-curated, raw tractograms (i.e., in an unsupervised fashion or using an unsupervised-learning technique), and a filtering threshold may be computed in the learned representation space using a set of labeled streamlines. Once training is over, a resulting learned latent space may be a low-dimensional robust representation of the input streamlines, where similar streamlines are located next to each other. The filtering of plausible and implausible streamlines may then be carried out by projecting to the latent space examples of reference streamlines. Moreover, the to-be-filtered streamlines may be projected to the latent space and labeled according to a classification technique (e.g., a K nearest-neighbors technique, where K is a non-zero integer; a generative model; a Fisher discriminator; a kernel method; a decision tree; a random forest; a boosting technique, a linear perceptron; and/or a multi-layer neural network). FINTA may provide superior performance (e.g., more accurate tractograms, such as an accuracy greater than 80 or 90%; a sensitivity of greater than 80 or 90%; a precision greater than 70 or 78%; and an F1-score of greater than 70 or 80%) relative to existing tractography techniques. In addition, FINTA may be linear in terms of the streamline count at test time, thereby providing faster tractogram filtering (i.e., faster analysis). For example, FINTA may significantly reduce the computation time needed to obtain revised or improved tractography results (which are sometimes referred to as ‘second tractography results’) relative to existing analysis techniques (e.g., by 10×), thereby reducing the use of processor, memory and communication resources in or associated with a computer system (such as computer system 1000 in FIG. 10).

Notably, in some embodiments, the autoencoder neural network may include an encoder neural network (which may be variational or not) and a decoder neural network. The output of the encoder neural network may be a so-called ‘latent space’. The autoencoder neural network may have been trained on a set of neurological fibers obtained by a tractography technique on MRIs associated with one or more brains. These MRIs may be optionally registered onto a reference space (such as or similar to the Montreal Neurological Institute or MNI space). Then, the neurological fibers of a reference bundle may be input to the encoder neural network and projected into the latent space. In this way, each neurological fiber may be encoded as a ‘latent vector’ lying in the latent space. These latent vectors may be defined as a set A. Next, given one or more MRIs of another individual (which may or may not be registered), the tractography technique may be used to recover an associated tractogram (e.g., a set of neurological fibers). The neurological fibers in this tractogram are then input to the encoder neural network and converted into second latent vectors. These second latent vectors may be defined as a set B. Moreover, a classification technique (such as a K nearest-neighbors technique, where K is a non-zero integer; a generative model; a Fisher discriminator; a kernel method; a decision tree; a random forest; a boosting technique, a linear perceptron; and/or a multi-layer neural network) may compare set A and set B. The latent vectors in set B that are close (in a hyperdimensional latent space) to at least one latent vector in set A may be kept (i.e., included in the second tractography results), and the remaining latent vectors may be excluded or eliminated.

In some embodiments of tractography in the analysis pipeline, the computer system may compute, using a predetermined autoencoder neural network, the second tractography results that specify a second set of neurological fibers based at least in part on tractography results provided by one or more prior operations or stages in the analysis pipeline and information associated with a neurological anatomical region. Note that the predetermined autoencoder neural network may be trained using an unsupervised-learning technique. Moreover, a subset of the set of neurological fibers may be anatomically implausible and the second set of fibers may exclude the subset. For example, a latent space provided by the autoencoder may distinguish anatomically plausible and anatomically implausible neurological fibers. Furthermore, the second set of neurological fibers may, at least in part, be different from the set of neurological fibers.

For example, the predetermined auto-encoded neural network may identify different types of bundles of neurological fibers, where the different types of bundles of neurological fibers include different integer numbers of neurological fibers. Moreover, the identifying may include: discarding one or more neurological fibers in the set of neurological fibers; or classifying the one or more neurological fibers as a given type of bundle of neurological fibers that includes an integer number of neurological fibers.

Furthermore, the computing may include filtering and grouping the second set of neurological fibers from the set of neurological fibers based at least in part on the information associated with the neurological anatomical region. For example, the filtering may include classifying one or more neurological fibers as a type of bundle of neurological fibers comprising an integer number of neurological fibers.

Additionally, the computing may include: transforming (e.g., using the predetermined autoencoder) the set of neurological fibers to a latent space having a smaller number of dimensions than the set of neurological fibers; and classifying one or more neurological fibers as a type of bundle of neurological fibers that includes an integer number of neurological fibers based at least in part on a classification technique and a reference space encoded (e.g., by the predetermined autoencoder neural network) in the latent space that corresponds to the information associated with the neurological anatomical region. Note that the reference space may specify different types of bundles of neurological fibers that include different integer numbers of neurological fibers. Moreover, the classification technique may include: a K nearest-neighbors technique, where K is a non-zero integer; a generative model; a Fisher discriminator; a kernel method; a decision tree; a random forest; a boosting technique, a linear perceptron; and/or a multi-layer neural network (such as a classification neural network, e.g., one or more convolutional layers, one or more residual layers and/or one or more dense layer and/or one or more fully connected layers, which include a softmax activation function that generates an output).

In some embodiments, the computer system may sample the latent space based at least in part on a sampling technique; and generate neurological fibers in the second set of neurological fibers based at least in part on the sampled latent space. For example, the sampling technique may include: rejection sampling, a Metropolis-Hastings technique, Gibbs sampling, etc. This sampling technique may blindly sample the latent space or may be guided by a reference space or set of latent vectors. Note that the sampled latent space may result in new latent vectors, which may be input to the decoder neural network to generate new neurological fibers. These new neurological fibers may be included in or used to populate a preexisting tractogram.

Furthermore, the predetermined autoencoder neural network may include an encoder neural network (which may be variational or non-variational) and a decoder neural network, where a given neural network in the predetermined autoencoder neural network may include or combine one or more convolutional layers, one or more residual layers and one or more dense or fully connected layers. Additionally, a given node in a given layer in the given neural network may include an activation function that includes: a ReLU, a leaky ReLU, an ELU activation function, a parametric ReLU, a tanh activation function, a sigmoid activation function, and/or another type of activation function. Note that an output of the given neural network may or may not include an activation function, such as: a tanh activation function, a sigmoid activation function, an identity function, and/or another type of activation function.

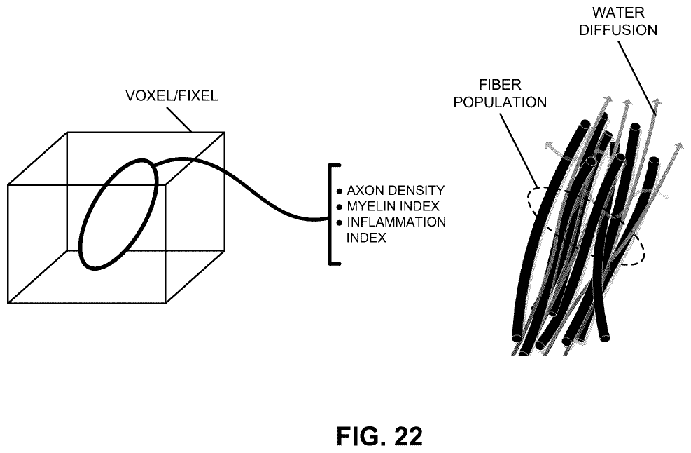

FIGS. 7 and 21-22 present a drawing and images illustrating examples of tractography. As shown in FIG. 22, tractography may involve a track-specific tissue assessment, and accurate tractography may enable assessment of one or more attributes or characteristics associated with the microstructure environment of each fiber population (such as an axon density index, a myelin index, an inflammation index, etc.).

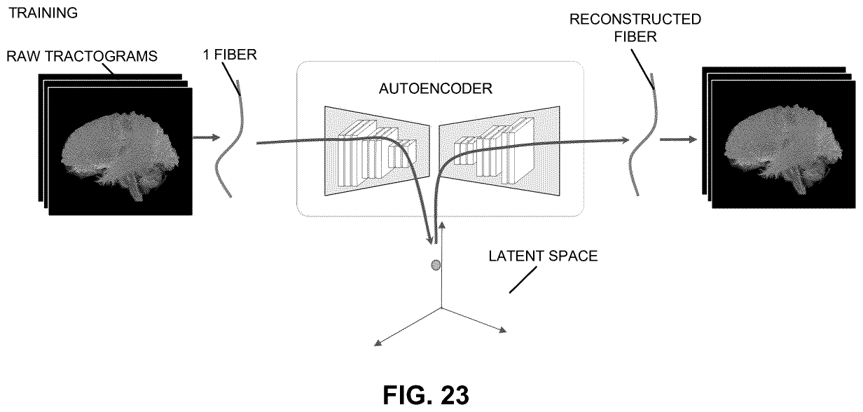

Autoencoder neural networks are deep neural networks that may be able to learn an efficient representation of data in an unsupervised fashion using a dimensionality reduction approach. Autoencoder neural networks may be trained to reconstruct an input signal through a series of compressing operations (which are referred to as the ‘encoder’), followed by a series of decompression operations (which are referred to as the ‘decoder’). As shown in FIGS. 23-29, between the encoder and the decoder there is the so-called latent space, where each point is an encoded representation of an input data sample. As discussed previously, in the analysis techniques, the input data may be a streamline, which is a sequence of three-dimensional points. Notably, a streamline may be an ordered sequence of points s={c₁, c₂, . . . , cₙ}, where cᵢ is a real number in a 3D space, that represents a package of similarly oriented neurological fibers describing a neural pathway within the brain white matter. A tractogram representing a set of M streamlines may be expressed as T={S₁, S₂, . . . , Sₘ}.

Autoencoder neural networks may offer advantages over other types of neural networks. First, they may be trained using raw unlabeled data, which is an asset in the context of brain tractography. Second, because the latent representation may be obtained by a series of compressing operations, two neighboring points in the latent space may correspond to input data instances that share similar characteristics. Consequently, the encoded representation may be used to redefine the notion of inter-data distance. In the context of tractography, instead of measuring the distance between two streamlines with a geometric distance function, one can project the streamlines into the latent space and measure, e.g., their Euclidean distance.

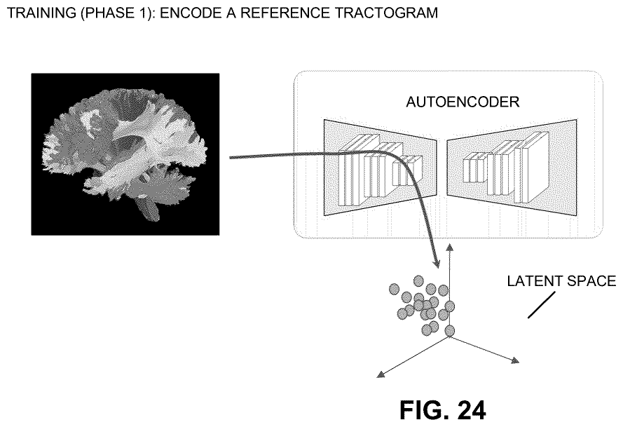

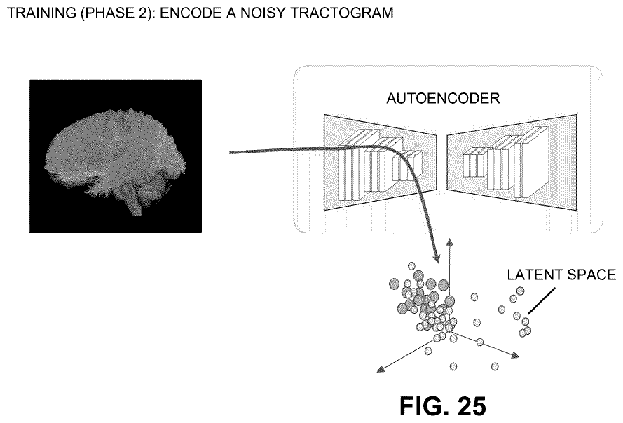

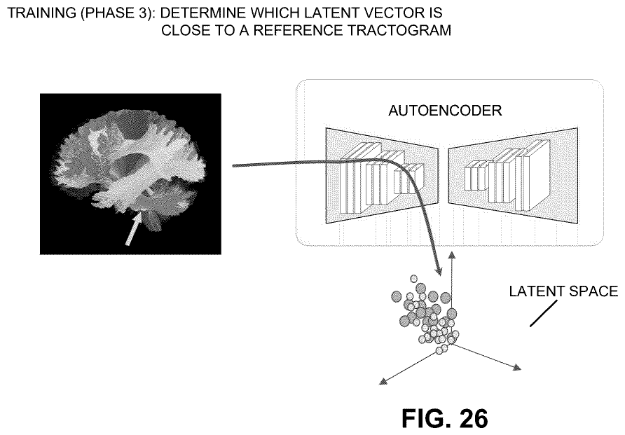

As shown in FIGS. 23-29, which present drawings illustrating examples of a cleaning and bundling operation, the disclosed analysis technique may include the following operations. First, an autoencoder neural network may be trained with a raw, uncurated tractogram. Based at least in part on the training tractogram, streamlines of interest may be labeled. These streamlines may be examples of anatomically plausible neurological fibers, streamlines representing neurological fibers belonging to predefined sets of bundles, or other streamlines that are of interest. Moreover, the streamlines may be labeled as positive streamlines. (Note that the autoencoder neural network may be trained in an unsupervised manner. However, the process of selecting the correct ‘neurological’ fibers, e.g., using a latent space and a nearest-neighbor technique, may use a target, such as labeled data.) Then, as shown in FIG. 24, the positive streamlines may be projected into the latent space with the encoder neural network. (In FIGS. 23-29, the latent vectors are illustrated by the circles.) Note that in case of more than two classes (such as for a multi-label bundling operation), streamlines of several classes may also be projected into the latent space. Next, as shown in FIGS. 25 and 26, given a new tractogram that is to be filtered, the streamlines of this tractogram are projected into the latent space and are labeled according to the distance to their nearest neighbor. Consequently, the filtering process in the disclosed analysis techniques may take place in the latent space.

In some embodiments, the nearest-neighbor discrimination may employ a latent-space distance cut-off value (which is sometimes referred to as a ‘filtering threshold’) relative to a (labeled) reference set of streamlines to discriminate the uncurated streamlines of a tractogram. The filtering threshold may represent a minimum distance at which a streamline is considered to be implausible, and its value may be computed on the separate reference set of streamlines. For example, the filtering threshold may be determined from an optimal point in a receiver operating characteristic (ROC) curve that evenly rewards true positives and penalizes false positives. Alternatively, the filtering threshold may be determined using data from a variety of tracking settings (e.g., probabilistic, deterministic, global, etc.) and datasets. Note that the filtering threshold may be, e.g., 6.568 or 13.634. However, in general the filtering threshold may be tailored or selected for a particular dataset being analyzed.

Moreover, the autoencoder neural network may be a fully convolutional neural network whose overall structure is summarized in Table 1. This autoencoder neural network may accept a streamline at its input. Because raw streamlines have a different number of vertices (long streamlines have more vertices than shorter ones), in the analysis techniques streamlines may be resampled so that they have an equal number of vertices (e.g., 256 in Table 1). Note that the latent space length may be fixed at, e.g., a value smaller than 512, such as 32. However, other values may be used.

TABLE 1

| Part | Type | Features | Size |
|---|---|---|---|
| Input | — | — | 3;256 |
| Encoder | 1D convolution | 32 | 256 |
| | 1D convolution | 64 | 128 |
| | 1D convolution | 128 | 64 |
| | 1D convolution | 256 | 32 |
| | 1D convolution | 512 | 16 |
| | 1D convolution | 1024 | 8 |
| Latent Space | Fully Connected | 32 | — |
| Decoder | Upsampling + 1D | 1024 | 8 |
| | convolution | | |
| | Upsampling + 1D | 512 | 16 |
| | convolution | | |
| | Upsampling + 1D | 256 | 32 |
| | convolution | | |
| | Upsampling + 1D | 128 | 64 |
| | convolution | | |
| | Upsampling + 1D | 64 | 128 |
| | convolution | | |
| | Upsampling + 1D | 32 | 256 |
| | convolution | | |
| Output | — | — | 3;256 |

Furthermore, the autoencoder may be trained with an Adam optimizer with a mean squared-error loss. The hyperparameters may be adjusted using a Bayesian search technique. The learning rate of the optimizer may be fixed to a value of 6.68×10⁻⁴, and weight decaying regularization with a 0.13 valued parameter may be used. Note that Table 1 provides an example of an autoencoder neural network structure. The encoder neural network may use strides of size 2, and the decoder neural network may use strides of size 1. Additionally, the upsampling stages in the decoder neural network may use an upsampling factor of 2. The autoencoder neural network may use ReLU activations throughout its convolutional layers.

Note that latent space of the autoencoder neural network may allow the streamline pair-wise distance to be reinterpreted. Notably, the analysis techniques may use the Euclidean distance between the latent representation of two streamlines as a proxy to measure their structural similarity. However, in other embodiments, a different similarity metric may be used, such as a Manhattan distance.

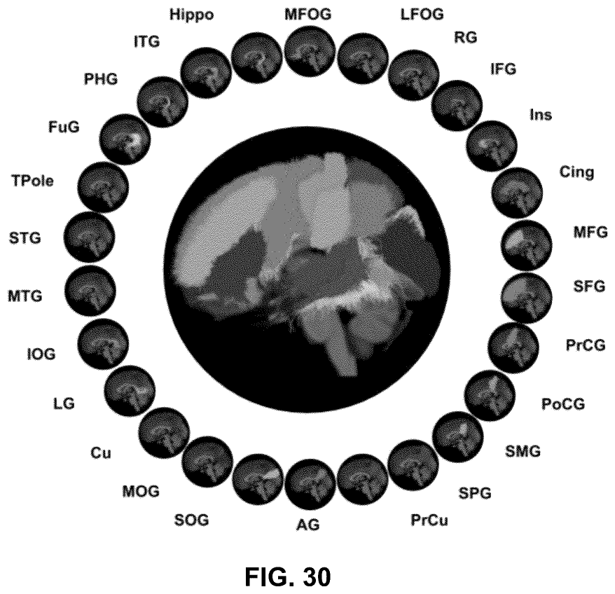

FIG. 30 presents a drawing illustrating an example of bundling results in different neurological anatomical regions.

In contrast with existing filtering techniques that assume that the estimated streamline population at every voxel is proportionally supported by the diffusion data, the disclosed autoencoder neural network may work on tracking data only. This may make the tractography analysis techniques less sensitive to or immune against domain adaptation issues. Furthermore, in comparison with a quadratic or super-quadratic complexity of some existing filtering techniques, the disclosed tractography analysis techniques may be linear in terms of the streamline count at test time.

Additionally, compared to other deep learning techniques that may be specifically oriented to classification tasks (e.g., regular classification convolutional neural networks), the disclosed tractography analysis techniques may provide the benefit of using an unsupervised learning approach. Thus, the disclosed tractography analysis techniques may not depend on the number of classes in the input data as the output of the network does not look for maximizing the probability of a given class among the possible ones. Consequently, the disclosed tractography analysis techniques may be better suited to the reality of tractography, where there is a limited knowledge about the ground truth, and where the analysis may lend itself to different organizational levels and, thus, different classification degrees. Therefore, the disclosed tractography analysis techniques may naturally adapt to new sets of classes without the need of having to retrain the autoencoder neural network.

A potential side-benefit of the disclosed tractography analysis techniques is that the reconstructed streamlines describe a locally smooth (yet ‘denoised’) trajectory. Succeeding analysis pipeline or visualization processes may benefit from this attribute, thereby providing a less complicated or a more realistic long-range apparent fiber trajectory representation.

In some embodiments, downstream tractography tasks may benefit from filtering strategies applied at earlier stages when performed in a coherent manner. For example, one of such tasks is the structural connectivity analysis (or connectomics). Because the disclosed tractography analysis techniques more accurately reject implausible streamlines, they may preserve the existing connectivity. Consequently, the disclosed tractography analysis techniques may regenerate the connectome to a high degree of accuracy.

Moreover, the disclosed tractography analysis techniques may be robust across a varying spectrum of settings. The disclosed tractography analysis techniques assume that, provided that the implausible streamlines have overall distinctive features from the plausible ones, they may be cast to different regions in the latent space, and thus the filtering framework may be able to separate them, regardless of their location and arrangement in the native space or within-fascicle balance. Similarly, the autoencoder neural network may have the ability to disentangle a varying number of bundles or streamline groups. Note that the disclosed tractography analysis techniques may be used with datasets having a wide range of values for a ratio of plausible streamlines to implausible streamlines, such as between 5-70.

Continuing the discussion of the analysis technique, during the microstructure analysis, information at a sub-millimeter resolution (and, thus, less than 1 mm³ voxel) may be determined, such as: fractional anisotropy, free water, etc. For example, information may be inferred at a higher resolution from lower-resolution measurements by solving an inverse nonlinear problem to determine parameters in or associated with the voxels. In some embodiments, the microstructure analysis may determine an index or a metric that reflects changes to: myelined axons, microglia, astrocytes, lesion edema, cebrebrospinal fluid, a characteristic or attribute associated with microstructure environment, etc. In some embodiments, the microstructure analysis may use: diffusion tensor imaging, diffusion component imaging and/or diffusion-basis spectrum imaging.

Moreover, during the region-wise microstructure statistics analysis, microstructure maps in multiple images (such as more than five images) may be used to determine statistics for neurological anatomical regions (e.g., relative to controls in a brain atlas) for parameters, such as apparent fiber density, free water, etc. (which may include one or more biomarkers in a set of white-matter disease biomarkers). In some embodiments, the region-wise microstructure statistics may be computed using a histogram or a fit to a distribution (such as a chi-square distribution, a Student's t-distribution, a Gaussian distribution, etc.) of correlations between different regions. Note that the region-wise microstructure statistics may specify or indicate pathology, such as: disease, disease progression, risk for disease or disease progression, latency of disease, etc. As shown in FIG. 30, in some embodiments the neurological anatomical regions may include predefined neurological anatomical regions associated with functional regions in grey matter.

Furthermore, during the region-wise statistical analysis, analysis may be performed using the statistics for one or more parameters or indexes in one or more different neurological anatomical regions to compute one or more aggregate indexes or metrics (such as one or more biomarkers in a set of white-matter disease biomarkers). Note that this analysis may be performed using a predefined model, such as a machine-learning model (with one or more thresholds) or a neural network.

Additionally, as described further below with reference to FIGS. 8 and 34-42, the determined indexes, metrics and/or statistics (such as the set of white-matter disease biomarkers) may be used on the diagnostic or treatment recommendation operation or TRO (and, more generally, a feedback operation) for a group or population or for an individual.

As discussed previously, in some embodiments the computer system may use an analysis model that is pretrained or predetermined using a machine-learning technique (such as a supervised learning technique, an unsupervised learning technique and/or a neural network) and a training dataset. For example, the analysis model may include a classifier or a regression model that was trained using: a support vector machine technique, a classification and regression tree technique, logistic regression, LASSO, linear regression, a neural network technique (such as a convolutional neural network technique, an autoencoder neural network or another type of neural network technique) and/or another linear or nonlinear supervised-learning technique.

For example, FIG. 31 presents a drawing illustrating an example of a skull stripping neural network configuration in an analysis pipeline. In FIG. 31, A1 represents a 3D convolution operation, followed by an instance normalization operation, followed by leaky ReLU operation, followed by a 3D convolution operation, followed by an instance normalization operation, and then followed by a leaky ReLU operation. Moreover, B1-4 represents instances of a 3D convolution of size/2 operation, followed by an instance normalization operation, followed by leaky ReLU operation, followed by a 3D convolution operation, followed by an instance normalization operation, and then followed by a leaky ReLU operation. Furthermore, C1-4 represents instances of a 3D convolution operation, followed by an instance normalization operation, followed by leaky ReLU operation, followed by a 3D convolution operation, followed by an instance normalization operation, and then followed by a leaky ReLU operation. Additionally, D1-4 represent instances of a 3D convolution transpose of size 2× operation, followed by a feature maps concatenation and a 3D convolution operation, followed by an instance normalization operation, a leaky ReLU operation, followed by a 3D convolution operation, followed by an instance normalization operation, and then followed by a leaky ReLU operation. E1-4 represents instances of a 3D convolution operation, and then followed by a 3D convolution transpose operation (repeated N times, where N is a non-zero integer). Note that in FIG. 31 the loss function may include: cross-entropy, a dice score and regularization.

Moreover, FIG. 32 presents a drawing illustrating an example of a structural MRI segmentation neural network configuration. In FIG. 32, A1 represents a 3D convolution operation, followed by an instance normalization operation, followed by leaky ReLU operation, followed by a 3D convolution operation, followed by an instance normalization operation, and then followed by a leaky ReLU operation. Moreover, B1-4 represents instances of a 3D convolution of size/2 operation, followed by an instance normalization operation, followed by leaky ReLU operation, followed by a 3D convolution operation, followed by an instance normalization operation, and then followed by a leaky ReLU operation. Furthermore, C1-4 represents instances of a 3D convolution operation, followed by an instance normalization operation, followed by leaky ReLU operation, followed by a 3D convolution operation, followed by an instance normalization operation, and then followed by a leaky ReLU operation. Additionally, D1-4 represent instances of: a 3D convolution transpose of size 2× operation; or a 3D convolution transpose of size 2× operation, followed by a feature maps concatenation and a 3D convolution operation, followed by an instance normalization operation, a leaky ReLU operation, followed by a 3D convolution operation, an instance normalization operation, and then followed by a leaky ReLU operation. E1-4 represents instances of a 3D convolution operation, and then followed by a 3D convolution transpose operation (repeated N times, where N is a non-zero integer). Note that in FIG. 32 the loss function may include: cross-entropy, a dice score, a focal loss and regularization.

Furthermore, FIG. 33 presents a drawing illustrating an example of a dMRI segmentation neural network configuration in an analysis pipeline. In FIG. 33, A1 represents a 3D convolution operation, and then followed by a ReLU operation. Moreover, B1-4 represents instances of a 3D convolution of size/2 operation, and then followed by a ReLU operation. Furthermore, C1-9 represents instances of a four-layer dense block (which may include a neural network module that connects several convolution layers, e.g., 4 to 6, with each other via a series of feature map concatenation operations). Additionally, D1-4 represent instances of: a 3D convolution transpose of size 2× operation; or a 3D convolution transpose of size 2× operation, followed by a ReLU operation, followed by a feature maps concatenation and a 3D convolution operation, and then followed by a ReLU operation. Note that in FIG. 33 the loss function may include: cross-entropy, a dice score and regularization.

We now describe the set of white-matter disease biomarkers and their use in conjunction with diagnosis, monitoring and treatment of Alzheimer's disease, which is used as an illustrative example. However, these analysis techniques may be used with a wide variety of diseases.

Alzheimer's disease is usually associated with grey matter, including: memory loss, brain atrophy and, more recently, loss of hippocampus volume, which is the memory center of the brain. However, very little attention has been given to white matter in Alzheimer's disease progression, both in academia and industry. This is, at least in part, a result of technological barriers to imaging and quantifying white matter, in animal models and humans. With recent advances in imaging technology (such as in microscopy, MRI, PET, etc.), and especially DMRI, it is now possible to quantify white-matter microstructure and its deterioration due to a neurodegenerative disease.

But why study white matter? Referring back to FIG. 8, a large body of scientific literature has shown changes in white matter with Alzheimer's disease, such as: microglia activation, loss of oligodendrocytes, demyelination, axonal loss and vascular degeneration. It is now clear that the scientific community must look beyond the neuron and the cortex of the medial temporal lobe (where the hippocampus is located) and focus attention on white-matter integrity.

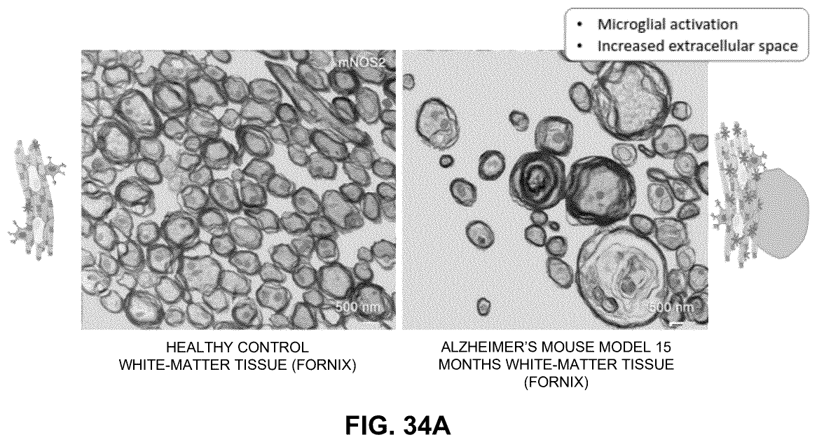

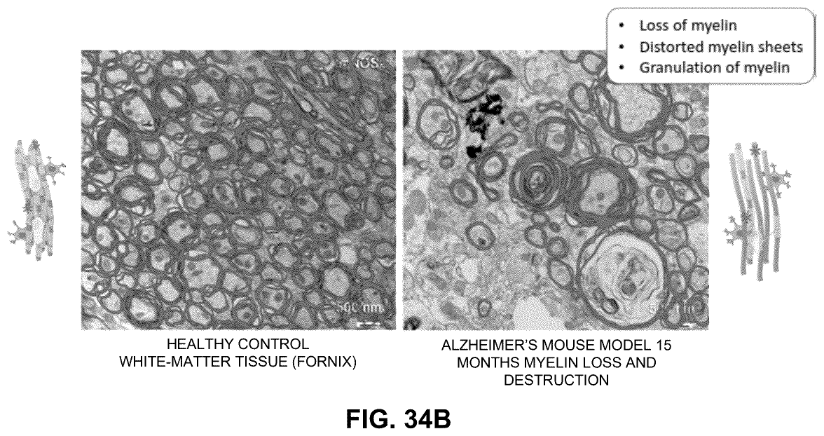

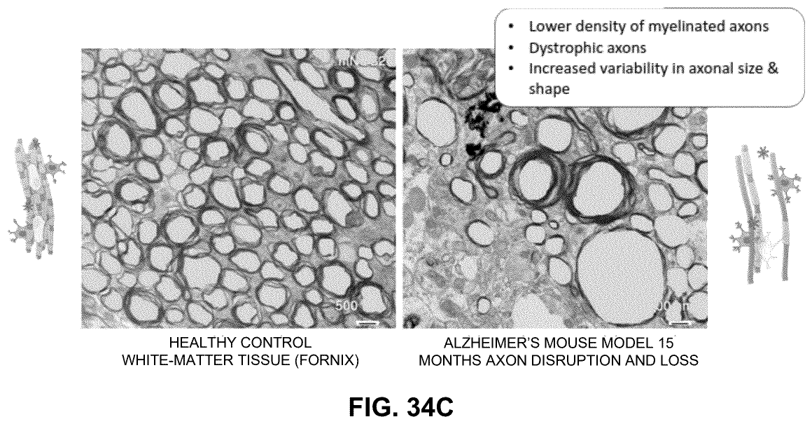

White-matter abnormalities have been identified in several animal studies using high-resolution microscopy, histology and different staining techniques to highlight myelin, axons, and the extracellular space. Notably, studies of 15 months old Alzheimer's disease mice clearly highlight abnormalities in the white matter. For example, as shown in FIG. 34A, which presents an image of white-matter axons in an animal study for healthy controls and Alzheimer's disease on histology illustrating free water and neuroinflammation, there are increased extracellular space and microglia activation because of neuroinflammation. Moreover, as shown in FIG. 34B, which presents an image of white-matter axons in an animal study for healthy controls and Alzheimer's disease illustrating axon myelin degradation, there are loss of myelin, and distortion and granulation of the myelin sheets, which indicate myelin degradation or demyelination. Furthermore, as shown in FIG. 34C, which presents an image of white-matter axons in an animal study for healthy controls and Alzheimer's disease illustrating axon swelling, there are lower densities of myelinated axons, dystrophic axons, axon swelling and variability in size and shape of axons, which indicate axon disruption or axonal loss.

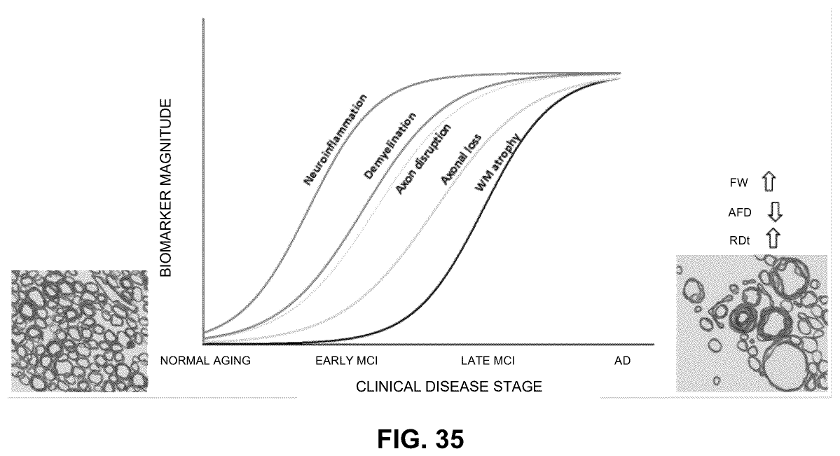

Based at least in part on small animal and human studies, the progression of white-matter deterioration with Alzheimer's disease is now understood to be the result of a cascade of microstructural events. As shown in FIG. 35, which presents a drawing illustrating white-matter disease progression from a healthy control, to early/late mild cognitive impairment, and then to Alzheimer's disease, this process starts with neuroinflammation, followed by oligodendrocyte dysfunction, demyelination, axonal disruption and, ultimately, the death in the white matter, which causes white-matter atrophy and gray-matter atrophy. Note that the free water and the free water-corrected radial diffusivity (RDt) increase (conversely, the inverse free water-corrected RDt decreases), while the apparent fiber density decreases.

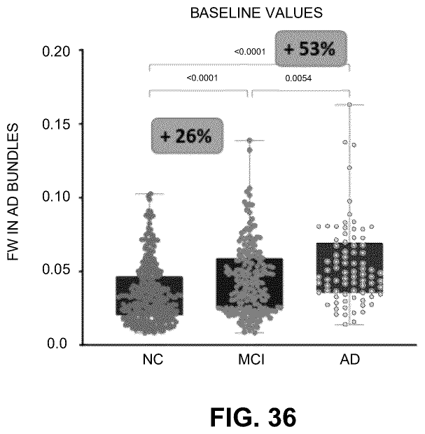

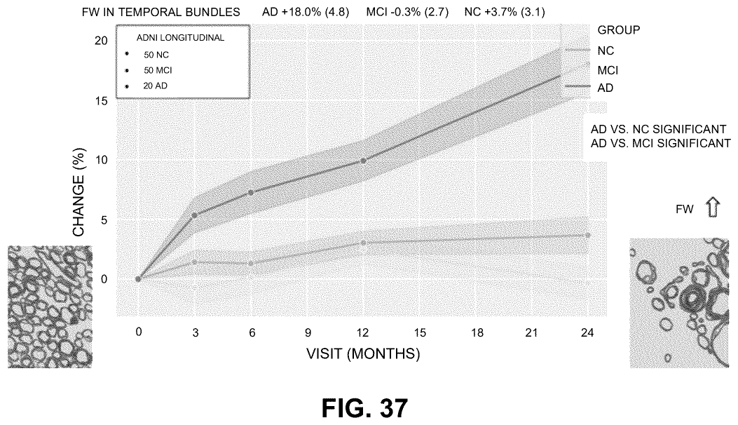

As discussed previously, the set of white-matter disease biomarkers for Alzheimer's disease may include white-matter microstructure markers of neuroinflammation, demyelination and/or axonal disruption. Notably, the set of white-matter disease biomarkers computed using the analysis techniques may include free water, which is an indirect measure of neuroinflammation. The amount of free water may be very different between normal or healthy controls (NC), mild cognitive impairment (MCI), and Alzheimer's disease patients. For example, based at least in part on 500+ participants in the Alzheimer's disease neuroimaging initiative (ADNI) database, we have found that patients with mild cognitive impairment have 26% more free water than normal controls, while Alzheimer's disease patients have 53% more free water than normal controls. This is shown in FIG. 36, which presents a drawing illustrating an example of free water in white-matter bundles in three groups of subjects, including healthy controls, mild cognitive impairment and Alzheimer's disease. Moreover, free water also dramatically increases as a function of time for Alzheimer's disease patients (an 18% increase) compared to mild cognitive impairment and normal controls, which appear to be stable as a function of time. This is shown in FIG. 37, which presents a drawing illustrating an example of free-water change as a function of time in white-matter bundles in the three groups of subjects. Furthermore, free water appears to be a good biomarker to discriminate Alzheimer's disease patients. Once again, based at least in part on 500+ ADNI participants, receiver operator characteristic analysis indicates that an individual with a free-water value greater than or equal to 0.04 has a 75% probability of being diagnosed with Alzheimer's disease.

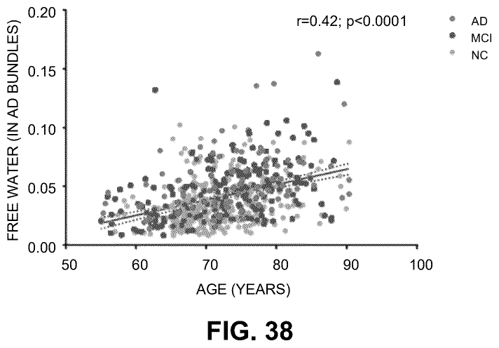

Moreover, free water is correlated with age, which is an important risk factor for Alzheimer's disease. Indeed, statistics show that, at the age of 85 years old, a person has one in three probability of suffering from Alzheimer's disease. This is shown in FIG. 38, which presents a drawing illustrating an example of correlation between free water and age.

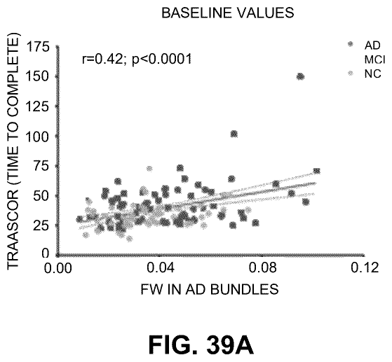

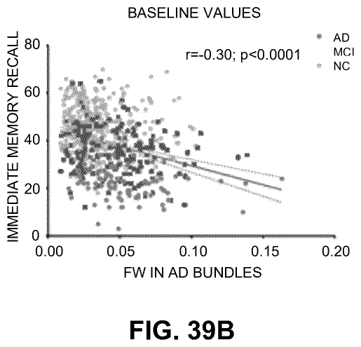

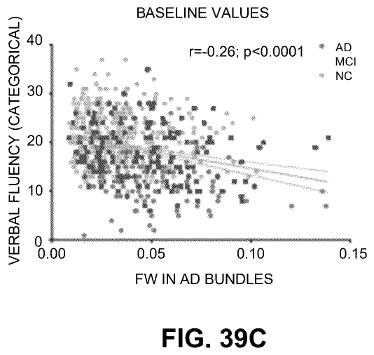

Furthermore, free water is very well anti-correlated with cognition. Notably, the Alzheimer's Disease Assessment Scale-Cognitive Subscale (ADAS-Cog13), an overall and global cognitive score is anti-correlated with free water. Stated differently, people performing poorly on this test have more free water (neuroinflammation) in their white matter. Similar associations are seen with processing speed, episodic memory, and fluency. These are shown in FIGS. 39A-C, which present drawings illustrating examples of associations between free water and cognitive tests. These results, therefore, suggest that free water may be a good biomarker, e.g., to select participants to enroll in clinical trials, depending on the drug effect and the group targeted. For example, in a clinical trial, an anti-inflammatory drug may be administered to patients that have a free water value above, e.g., 0.1 (10%), which is indicative of patients that have too much neuroinflammation.

Additionally, free water may be a good biomarker for diagnosis, tracking disease progression and/or guiding treatment (such as a choice of medication and/or a dose). Notably, free water may serve as a biomarker to make sure a particular treatment does not increase neuroinflammation, but instead stabilize it or even decrease it over time. For example, an amyloid-clearing drug that effectively clears amyloid proteins from the brain, but that also increases neuroinflammation because of collateral damage, may not be a suitable treatment for affected patients.

The set of white-matter disease biomarkers for Alzheimer's disease may include demyelination. Demyelination may be indirectly measured by: the inverse free water-corrected radial diffusivity; other MRI myelin-specific markers (such as: T₁/T₂, a magnetization transfer ratio, etc.); and/or a myelin water fraction, which is the ratio of the area in the T₂ distribution (between 10 and 40 ms for humans) to the area of the entire T₂ distribution.

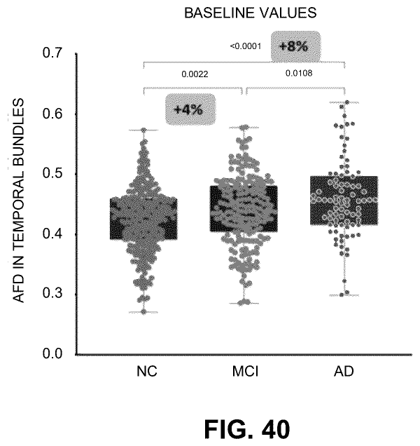

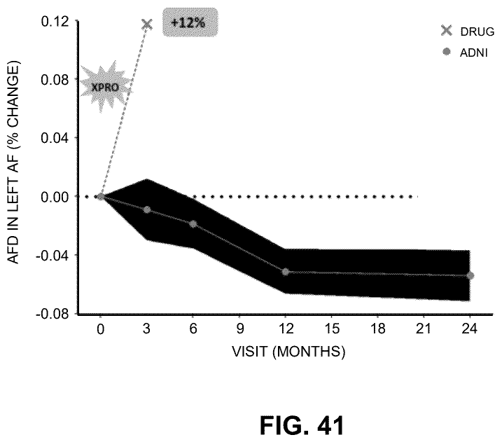

Moreover, the set of white-matter disease biomarkers for Alzheimer's disease may include apparent fiber density, which is an indirect measure of axonal disruption and quality/tissue repair. Note that apparent fiber density may be slightly different for different disease stages. As shown in FIG. 40, which presents a drawing illustrating an example of apparent fiber density in the three groups of subjects, Alzheimer's disease patients may have apparent fiber density decrease 2-4% in time, which indicates axon disruption. In contrast, normal controls may have stable apparent fiber density over time. Furthermore, patients with mild cognitive impairment may have an inverted U-shape curve, which has been previously observed for other imaging biomarkers for these patients. This may be explained by axon swelling and the variable size and shape of axons that occur as white-matter disease progresses (see, e.g., FIG. 34B), which may be occurring in the mild-cognitive impairment group before axonal loss. Apparent fiber density may be very specific to white-matter changes and may be robust to crossing neurological fibers, as well as partial volume from other tissue. Additionally, apparent fiber density may be the biomarker of choice that a drug needs to stabilize (or even increase) in order to show tissue repair, better axon quality and potential remyelination. This is shown in FIG. 41, which presents a drawing illustrating an example of a drug that increases the apparent fiber density of a patient with Alzheimer's disease. Note that the drug: increased the apparent fiber density as a function of time (34/35 bundles, with an average increase of 8%). Moreover, for ADNI bundles, the arcuate fasciculus (AF), the inferior fronto-occipital fasciculus (IFOF) and the inferior longitudinal fasciculus (ILF) increased by 11%.

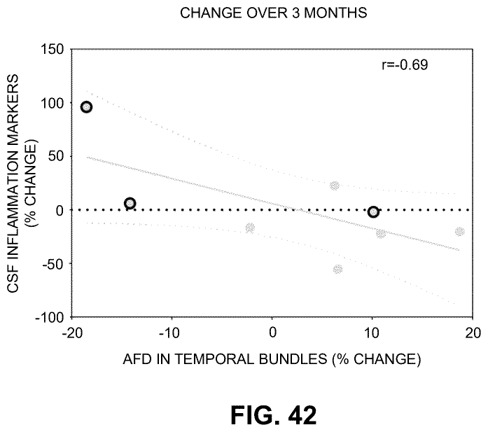

As shown in FIG. 42, which presents a drawing illustrating an example of associations between apparent fiber density and cerebrospinal-fluid inflammatory markers, apparent fiber density is strongly correlated with cerebrospinal-fluid inflammatory markers. This shows that, the more inflammation is reduced, the healthier the axons (as indicated by apparent fiber density). Note that apparent fiber density has also been shown to correlate with traumatic brain injury (e.g., in professional football players).

In some embodiments, the combination of the hippocampus volume with the set of white-matter disease biomarkers (such as the neuroinflammation, demyelination and/or axonal disruption metrics) helps improve the correlation score with Alzheimer's disease progression. Moreover, one or more of the set of white-matter disease biomarkers may be computed on a per-voxel basis and/or a per-neurological-fiber or fixel basis. In these ways, diseases can be characterized based at least in part on their overall effect on the white matter, on sub-regions or neurological anatomical regions of the brain (such as the corpus callosum, inferior fronto-occipital fasciculus and arcuate fasciculus for Alzheimer's disease, motor tracts for Parkinson's disease, and the medial temporal lobe for mild cognitive impairment), on a per-voxel and/or on a per-fixel basis.

The set of white-matter disease biomarkers may allow the computer system to determine the stage of a disease. As such, for a given Alzheimer's disease patient at a particular time, a physician may prescribe an anti-inflammatory drug, a remyelinating drugs, or a stabilization drug depending on the neuroinflammation, demyelination and/or axonal disruption scores of metrics for this patient. Similar feedback information may be provided by the computer system for one or more other neurological diseases, such as: Parkinson's disease, multiple sclerosis, Amyotrophic lateral sclerosis, etc.

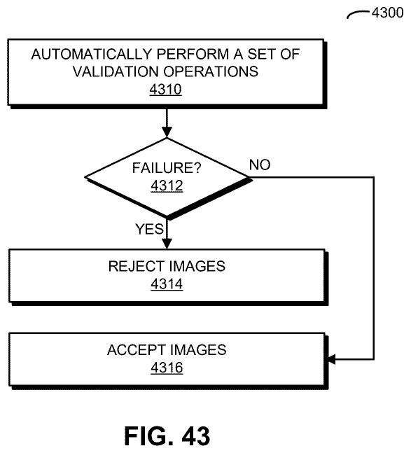

We now further describe additional embodiments of the QC techniques. Referring to FIG. 10, in some embodiments one or more of computers 1010 in computer system 1000 may perform the QC techniques. Notably, as discussed previously, existing QC techniques often suffer from a number of problems. Moreover, as described further below with reference to FIGS. 43-51, in order to address these challenges computer system 1000 may perform the QC techniques. During the QC techniques, one or more of optional control modules 1018 may divide the QC among computers 1010. Then, a given computer (such as computer 1010-1) may perform at least a designated portion of the QC. In particular, computation module 1014-1 may receive (e.g., access) information (e.g., using memory module 1016-1) specifying medical-imaging data that specify the central nervous system (including white matter) for one or more individuals. Note that the medical-imaging data may include or may correspond to structural MRI data and dMRI data. Then, computation module 1014-1 may automatically (i.e., without human decision-making or intervention) perform a set of validation operations in a QC pipeline. For example, as described further below with reference to FIGS. 43-51, the QC pipeline may include: performing QC on brain-tissue segmentation; performing QC on dMRI processing; performing QC on bundles determined from the images using a bundling technique involving tractography or atlas registration technique or a machine-learning technique; and performing QC on bundle-wise tractometry. Note that the QC techniques may include a pretrained neural network, and computation module 1014-1 may determine the bundles using the pretrained neural network. In some embodiments, a given validation operation may include computation module 1014-1 comparing the given validation operation with a given threshold associated with the given validation operation.

Note that the QC on the brain-tissue segmentation may include validating: a volume; a shape; a position; a comparison with a reference-atlas coordinate system; and/or detecting holes in a mask (such as a brain-tissue segmentation mask). Moreover, the QC on the dMRI processing may include validating: a range of DTI metrics; a range of HARDI metrics; and/or ranges of dMRI signals for an ODF and/or an fODF. Furthermore, the QC on the bundles may include validating: streamline statistics; bundle shape; bundle volume; and/or model comparisons. Moreover, the QC on the bundle-wise tractometry may include validating a range of DTI metrics; a range of Neurite Orientation Dispersion and Density Imaging (NODDI) metrics; a range of HARDI metrics and/or ranges of dMRI signals for an ODF and/or fODF measured along each bundle.

In some embodiments, the set of validation operations may include: validating metadata associated with the images; performing QC on a diffusion gradient; and/or performing QC on dMRI artifact correction. Note that the diffusion gradient may include: b-values, gradient sampling and/or a gradient configuration. Moreover, the dMRI artifact correction may include: motion detection, eddy-current-distribution detection and/or slice-outlier detection.

Additionally, the brain-tissue segmentation may include: segmenting the gray matter, gray matter sub-regions, the white matter, white matter sub-regions, the cerebrospinal fluid, and/or deep nuclei regions; and removing voxels that are not associated with brain tissue (such as eyes, bone, mouth, skin, etc.). In some embodiments, computation module 1014-1 may use the images associated with the structural MRI to extract the brain tissue. Then, using a remainder of the images associated with the dMRI, computation module 1014-1 may determine local models for voxels, where the local models indicate directions of water diffusion. Note that computation module 1014-1 may: compute diffusion metrics based at least in part on the local models; recover tracts in the brain tissue; and/or connect grey-matter regions using the bundles.

When the medical-imaging data fails one or more of the validation operations, computation module 1014-1 may reject the medical-imaging data. Otherwise, after passing the set of validation operations in the QC pipeline, computation module 1014-1 may approve and output the medical-imaging data for subsequent analysis (e.g., using an analysis pipeline) or one or more additional operations. Alternatively or additionally, in some embodiments computation module 1014-1 may raise a warning flag when an error has been detected, but is too small to reject the medical-imaging data. At the end, when too many warning flags have been raised (e.g. two or three), the medical-imaging data may be rejected. Alternatively or additionally, in some embodiments computation module 1014-1 may generate a QC report for the medical-imaging data, which may be stored in memory module 1014-1 and/or communicated to another electronic device using communication module 1012-1.

In these ways, computer system 1000 may automatically and accurately perform QC on the medical-imaging data. These capabilities may allow computer system 1000 to perform the subsequent analyses or one or more additional operations, such as: connectome analysis, white matter segmentation, one or more clinical trial enrollment or exclusion criteria, assessing the impact of a medical intervention for a disease (e.g., in a clinical trial for a candidate pharmaceutical agent, neurostimulation and/or another type of therapy), precision medicine (such as in selecting a correct medical intervention to treat a disease, e.g., as a companion diagnostic for a prescription drug or a dose of a prescription drug), etc. Consequently, the analysis techniques may facilitate accurate, value-added use of the measurement results, such as medical-imaging data.

We now describe embodiments of the method. FIG. 43 presents a flow chart illustrating an example of a method for automatically performing a set of validation operations, which may be performed by a computer system (such as computer system 1000 in FIG. 10). During operation, the computer system may automatically perform a set of validation operations (operation 4310), where, when one or more of the validation operations fails (operation 4312), the images are rejected (operation 4314). Otherwise, the images are approved or accepted (operation 4316). Note that the set of validation operations include: performing QC on brain-tissue segmentation; performing QC on dMRI processing; performing QC on bundles determined from the images using a bundle-assessment technique; and performing a bundle-specific tractometry QC on the dMRI metrics measured along the bundles. In some embodiments, the set of validation operations are performed sequentially.

The QC on the brain-tissue segmentation may include validating: a volume; a shape; a position; a comparison with a reference-atlas coordinate system; and/or detecting holes in a mask. Moreover, the QC on the dMRI processing may include validating: a range of DTI metrics; a range of HARDI metrics; and/or ranges of dMRI signals, e.g., for an ODF and an fODF. Furthermore, the QC on the bundles may include validating: streamline statistics; bundle volume; bundle shape; and/or model comparisons. Furthermore, the QC on the dMRI metrics measured along the bundles include validating: a range of DTI metrics; a range of Neurite Orientation Dispersion and Density Imaging (NODDI) metrics; a range of HARDI metrics; and/or ranges of dMRI signals, e.g., for an ODF and an fODF all measured along each bundles.

In some embodiments, the set of validation operations may include: validating metadata associated with the images; performing QC on a diffusion gradient; and/or performing QC on dMRI artifact correction. Note that the diffusion gradient may include: b-values, gradient sampling and/or a gradient configuration. Moreover, the dMRI artifact correction may include: motion detection, eddy-current-distribution detection and/or slice-outlier detection.

Furthermore, the set of validation operations may be implemented using a feed-forward pipeline.

Additionally, the brain-tissue segmentation may include: segmenting the gray matter, gray matter sub-regions, the white matter, white matter sub-regions, the cerebrospinal fluid, and/or deep nuclei regions; and removing voxels that are not associated with brain tissue (such as eyes, bone, mouth, skin, etc.). In some embodiments, the computer system may use the images associated with the structural MRI to extract the brain tissue. Then, using a remainder of the images associated with the dMRI, the computer system may determine local models for voxels, where the local models indicate directions of water diffusion. Note that the computer system may: compute diffusion metrics based at least in part on the local models; recover tracts in the brain tissue; and/or connect grey-matter regions using the bundles.

Moreover, the QC techniques as well as the image and data analysis methods used within the QC modules may include a pretrained neural network, and the computer system may determine the bundles using the pretrained neural network.

Furthermore, a given validation operation may include comparing the given validation operation with a given threshold associated with the given validation operation.

In some embodiments of method 4300, there may be additional or fewer operations. Furthermore, the order of the operations may be changed, and/or two or more operations may be combined into a single operation. For example, method 4300 may include an operation of providing a warning flag.

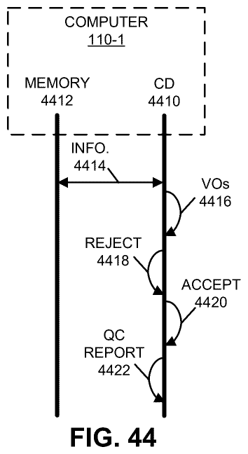

Embodiments of the analysis techniques are further illustrated in FIG. 44, which presents a drawing illustrating an example of communication among components in computer system 1000. In FIG. 44, a computation device (CD) 4410 (such as a processor or a GPU) in computer 1010-1 may access, in memory 4412 in computer 1010-1, information 4414 specifying medical-imaging data, such as dMRI and structural MRI. After receiving medical-imaging data, computation device 4410 may perform a set of validation operations (VOs) 1416 (which may be performed sequentially). When the medical-imaging data fails one or more of validation operations 1416, or get a too-large number of warnings, computation device 4410 may reject 1418 medical-imaging data. Otherwise, computation device 4410 may accept or approve 1420 medical-imaging data for additional analysis or processing. In some embodiments, computation device 4410 may automatically generate a QC report 4422 for the validation of the medical-imaging data and may store (e.g., in memory not shown) or communicate (e.g., using an interface circuit not shown) QC report 4422 to another electronic device (not shown).

While FIG. 44 illustrates communication between components using unidirectional or bidirectional communication with lines having single arrows or double arrows, in general the communication in a given operation in this figure may involve unidirectional or bidirectional communication.

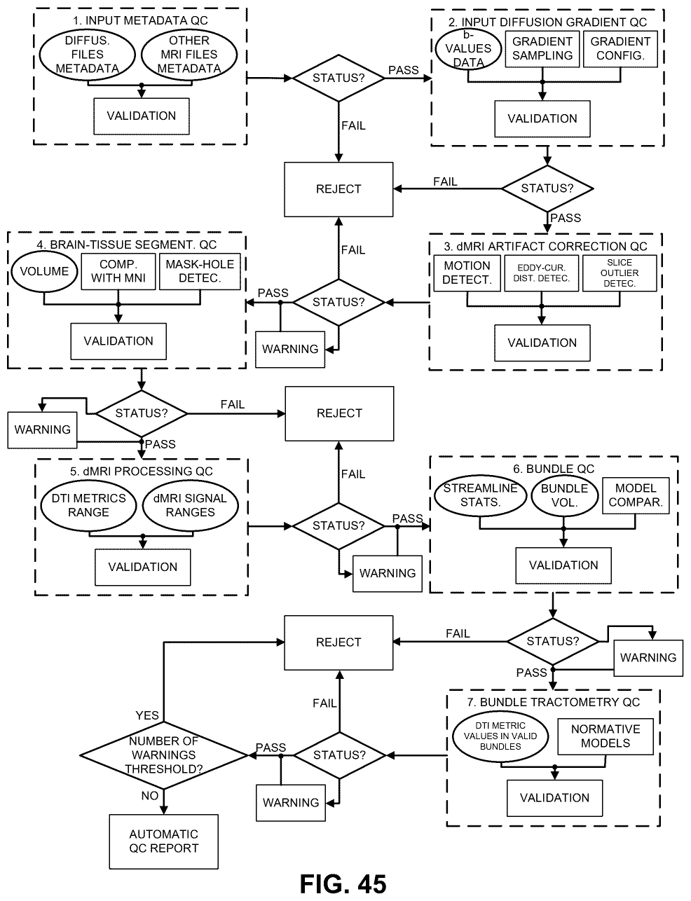

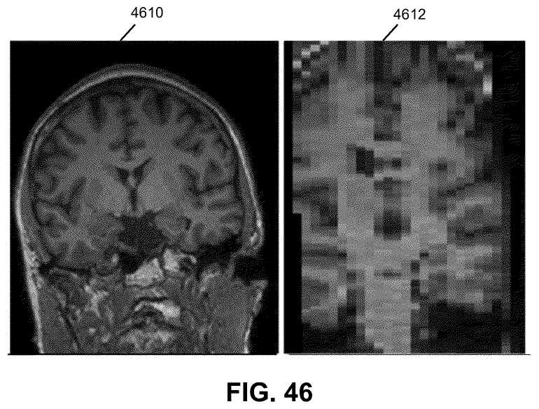

We now further describe the QC techniques. The disclosed QC techniques may implement a set of operations that span across the diffusion tractography and tractometry pipeline to detect erroneous data and incorrect intermediate results. Each time a QC operations fails, an error may be asserted, signaling that the output result could not be computed and/or may contain erroneous information. When no QC error is raised or asserted, the output may be assumed to be of sufficient quality to be presented to an end-user and/or used in subsequent analysis. The QC operations may also raise a warning flag. When a too large number of warning flags has been raised (e.g. more than two or three), this also signals that the output result could not be computed and/or may contain erroneous information. As shown in FIG. 45, which presents a flow chart illustrating an automated quality-control method, the disclosed QC techniques may include one or more of a set of validation or QC operation (which may be performed sequentially), including: validating metadata associated with the images; performing QC on a diffusion gradient; performing QC on dMRI artifact correction; performing QC on brain-tissue segmentation; performing QC on dMRI processing; performing QC on bundles determined from the images using a bundling method involving tractography, a registration technique or a machine learning method; and performing a bundle-specific tractometry QC on the dMRI metrics measured along the bundles. Notably, in input metadata validation, the goal may be to detect inconsistencies in the input images by direct inspection of their associated metadata. This may include image resolution that may be too large or too low; a slice thickness; an inter-slice distance that is too large or too low; and/or an echo time or repetition time of inappropriate length. This validation operation may also check a level of uniformity of the magnetic field. Note that these checks may be performed for every input image, such as T₁, T₂, proton density (PD), FLAIR, magnetization transfer ratio (MTR), inhomogeneous magnetization transfer (ihMT), MRI free water (MFW), and/or diffusion images. These images may have been acquired with or without a contrasting agent. FIG. 46 presents examples of a T₁ image 4610 with an appropriate resolution and an incorrect T₁ image 4612 with a low voxel resolution along the z axis.

Referring back to FIG. 45, QC on the diffusion gradient (such as the input diffusion gradient) may check if the b-values and the b-vectors have appropriate values and appropriate configurations. This may include checking for the presence of a b0 signal, because an absence of a b0 signal may trigger an error. Then, a check may be performed as to whether the non-zero b-values are within an appropriate range.

In some embodiments, the QC techniques may include checking whether the number of b-values and b-vectors is large enough to properly compute the selected local diffusion model. For example, at least six diffusion weighted images may need to be available in order to compute a diffusion tensor such as the diffusion tensor in Eqn. 5. Alternatively or additionally, for ODF or fODF models, at least 15 diffusion weighted images may need to be available.

Moreover, in some embodiments the QC techniques may include checking that the b-vectors are not collinear. This may be performed by computing the angle of each pair of the b-vectors and asserting an error when the angle is below a predetermined threshold (such as 1°).

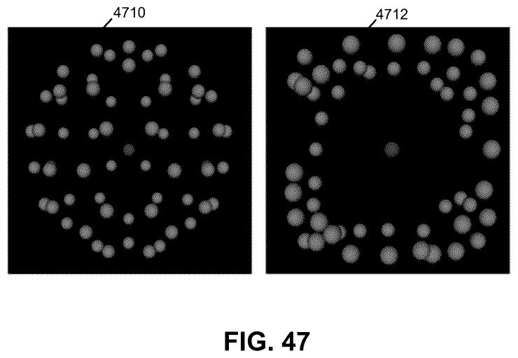

Note that the b-vectors of each shell may also need to be uniformly distributed on the sphere with a low discrepancy. This can be achieved in a variety of ways. For example, as shown in FIG. 47, which presents a drawing of two different single-shell diffusion MRI configurations, each b-vector may be defined as a point on a sphere and a spherical discrepancy, a spherical cap discrepancy, an L2 discrepancy on a spherical space and/or another discrepancy measure indicating how equi-distributed the points are on the sphere may be computed. An error may be asserted if the computed discrepancy exceeds a threshold τ. This threshold may be fixed (e.g., 0.05 for 20 b-vectors) or may decrease with the number of point N on the sphere, w.g., using a Lemieux technique:

$$τ = {τ}_{0} \frac{log {( N )}^{2}}{N} . \qquad ( 6 )$$

In some embodiments, a Delaunay triangulation may be performed over all non-collinear b-vector points. Then, the area of the triangles may be stored, and the average and standard deviation of the areas may be computed. If the resulting standard deviation is above a predefined threshold (e.g., 0.7 times the average triangle size), an error may be asserted. Alternatively or additionally, a machine-learning technique may be used to train a model (such as a classifier or a regression model) to discriminate between correct b-vector distributions (such as distribution 4710) from wrong distributions (such as distribution 4712). In FIG. 47, note that the red dots are b-vectors and the blue dots are the b0 signal. Moreover, in FIG. 47, b-vector distribution 4710 is uniformly distributed, while b-vector distribution 4712 is non-uniformly distributed.

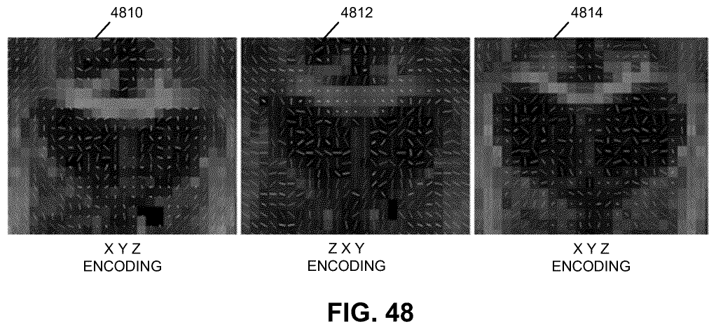

As discussed previously, the b-vectors may be unit vectors in the q-space. They may be established in the MRI acquisition protocol and written in the form of an XYZ table, where X, Y, and Z are the Euclidean axes. However, this table may be corrupted, which may cause permutations and flipping of the vector coordinates. Consequently, instead of XYZ encoding, the b-vector table may have a ZXY or −XYZ encoding that is apparent when looking at estimated fiber orientations such as DTI principal eigenvectors. FIG. 48 presents an image illustrating examples of a correct b-vector encoding 4810 and two incorrect b-vector encodings 4812 and 4814.

In order to detect invalid encoding, a fiber coherence index may be computed for the 24 possible b-vector configurations (including permutations and flipping) weighted by an anisotropy value (e.g., using the Schilling technique). The configuration corresponding to the maximum fiber coherence index may be the actual gradient encoding. In some embodiments, a supervised machine-learning segmentation model may be trained to detect invalid encoding.

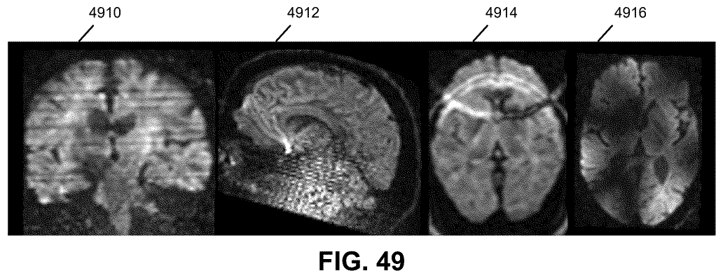

Furthermore, QC on dMRI artifact correction may detect diffusion MRI acquisition artifacts. This may include motion artifacts, which can occur when a patient moves during data acquisition. It may also include slice drops (such as axial, sagittal and/or coronal), inter-slice intensity variation, signal loss, checkerboard artifacts, and another type of aliasing artifacts. This is shown in FIG. 49, which presents an image illustrating examples of dMRI artifacts, such as: an inter-slice and intra-slice intensity artifact 4910, a checkerboard artifact 4912, chemical ring artifacts 4914, and signal loss artifacts 4916. These artifacts can be detected using a technique that measures the average motion between pair of diffusion MR images. In some embodiments, artifacts may be detected by measuring the amount of blur with and without a reference and/or using another MR image-assessment technique. Note that a supervised machine-learning model may be trained to discriminate good quality MR images from MR images that include artifacts.

Furthermore, motion artifacts (as well as other artifacts such as susceptibility artifacts, slice drops, etc.) can also be detected by segmenting the whole brain in the b0 image and all DWI volumes using a brain-tissue segmentation technique (which is typically used for ‘skull stripping applications’). This may result in a set of whole brain segmentation maps equal to the number of diffusion directions plus one. In the absence of motion, these maps are well aligned, typically yielding high DICE scores (e.g., >95%). However, motion artifacts are indicated when one or more segmentation maps are misaligned with the others, resulting in low DICE scores (e.g., <85%). Intermediate DICE values (e.g. between 85% and 95%) may trigger a warning flag, suggesting potential motion-related inconsistencies.

Additionally, QC on brain-tissue segmentation may detect erroneous brain-segmentation maps. These maps may include: a skull map; a gray-matter map; a gray-matter region map; a white-matter map; a white-matter region map; a cerebrospinal fluid map; a deep nuclei map; and/or a combination of two or more of these maps.

A brain-segmentation map may be obtained using an unsupervised segmentation technique, such as: K-Means, soft-K-Means, expectation maximization, another maximum likelihood, maximum a posteriori technique and/or a graph-based technique (such as graph cut or spectral clustering). The brain-segmentation map may also be obtained using an atlas-based registration process and/or by thresholding an MRI modality or an MRI feature image (such as a fractional anisotropy image).

Brain-segmentation maps may also be obtained using a supervised machine-learning segmentation technique. These techniques may implement a decision tree, a random-forest, boosting and/or a bagging technique. Deep neural networks may also be used, such as: a convolutional neural network, a single- or multi-layer perceptron, a transformer network, a recurrent neural network, a long short-term memory (LSTM) network, a neural network with an encoder and a decoder with and without skip connections, a back-end or a front-end network, and/or a combination of two or more of these neural-network techniques.

In some embodiments, brain-segmentation maps are obtained using software, such as: tools in the FMRIB software library (from Oxford University of Oxford, United Kingdom), e.g., FAST, BET, FIRST, etc.), FreeSurfer (from the Laboratory for Computational Neuroimaging at Harvard University of Cambridge, Massachusetts), FastSurfer, skull stripping (from the Poznan university of Technology of Poznan, Poland), Brain extraction in presence of abnormalities, using single and multiple MRI modalities or BrainMaGe (from the University of Pennsylvania of Philadelphia, Pennsylvania), Advanced normalization tools (ANTs-Atropos), and/or another type of software.

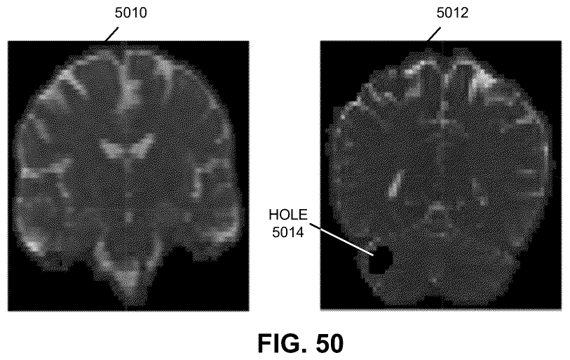

Brain-segmentation QC may be used to detect brain regions whose shape, volume or position is anatomically implausible, even for patients suffering from a disease. Anomalies may be detected when a brain region is too large or too small compared to a predetermined reference size. In some embodiments, implausible brain region shapes may be detected, such as regions with unusual holes, abnormal concavities or convexities. Abnormalities may also be detected based at least in part on a region connectivity analysis for when two or more regions are wrongly connected to each other or when a brain region has unusual disconnected components. FIG. 50 presents an image illustrating examples of a correct brain-segmentation map 5010 and an incorrect-brain segmentation map 5012 (which includes a hole 5014). Note that anomalies may be detected by comparing the brain-segmentation maps to those of a brain atlas and/or by using a supervised machine-learning model that is trained to differentiate between normal and abnormal brain region shapes.

In some embodiments, abnormal brain-segmentation maps may be detected using longitudinal analysis. In these embodiments, the shape, size and location of the brain regions of a given patient should be roughly the same for each brain image acquired during a study. Therefore, a deviation from this expected trend may indicate the presence of an anomaly.

Moreover, QC on the dMRI processing may include validating the integrity of the diffusion MRI models, such as the DTI or SD models. This QC operation may be performed by verifying estimated parameters of the models. Furthermore, the residual signal (which is the signal not explained by a given model) may be computed, and it may be verified that this residual signal does not exceed a predefined amount (e.g., 10 percent of the original signal). Note that this amount may be global for the entire brain or may be specific to each brain region, fixel or voxel depending on the characteristics of regions (e.g., cerebrospinal fluid, white matter or gray matter) that affect the diffusion models.

In some embodiments, the QC on the dMRI processing may include validating the metrics computed from the different models by making sure their values are within an anatomically plausible range. Note that the metrics may include: fractional anisotropy, mean diffusivity, radial diffusivity, axial diffusivity, DTI eigen values, free-water, any free-water corrected measures, apparent fiber density, ADC maps, attenuated ADC maps, a number of fiber orientations (NuFO), rotationally invariant spherical harmonics (RISH) features, intra cellular volume fraction (ICVF), and/or neurite orientation and dispersion density imaging (NODDI). These diffusion metrics may have been harmonized with a post-processing harmonization technique, such as ComBAT or another harmonization technique.

Note that anomalies may be detected when the values of these metrics in one or several voxels or fixels are outside a viable range determined by a minimum and maximum threshold. These thresholds may be specific for each metric (e.g., between 0.4 and 0.7 for fractional anisotropy) and may be computed from a normative reference population. This population may account for co-variables that affect these metrics, such as age, sex, and handedness. With a normative reference population, anomalies may be detected using a statistical test, such as: a z-test, a t-test, an ANOVA test, a Mann-Withney U-test, a Wilcoxon test, a Mann-Whitney Test, a Moods Median test and/or a Kruskal-Wallis test. Note that anomalies can be detected for the entire brain, for certain brain regions, or for certain voxels and/or fixels. In some embodiments, a supervised machine-learning model may be trained to detect or segment abnormal regions. Moreover, abnormal metrics may also be detected using longitudinal analysis. In these embodiments, the values of the metrics within a brain region, a voxel or a pixel of a given patient should be roughly the same during a study. Therefore, a deviation from this expected trend may indicate the presence of an anomaly.

Additionally, QC may be performed on the bundles determined from the images using a tractometry technique. The bundles may include groups of brain fibers having a similar shape and connecting the same gray matter regions. As mentioned previously, these brain fiber bundles may be extracted using a clustering technique, such as: QuickBundles, QuickBundlesX, Deep Fiber Clustering, RecoBundles, RecoBundlesX, TractSeg, XTRACT, FINTA, Deep WMA, and/or BINTA.

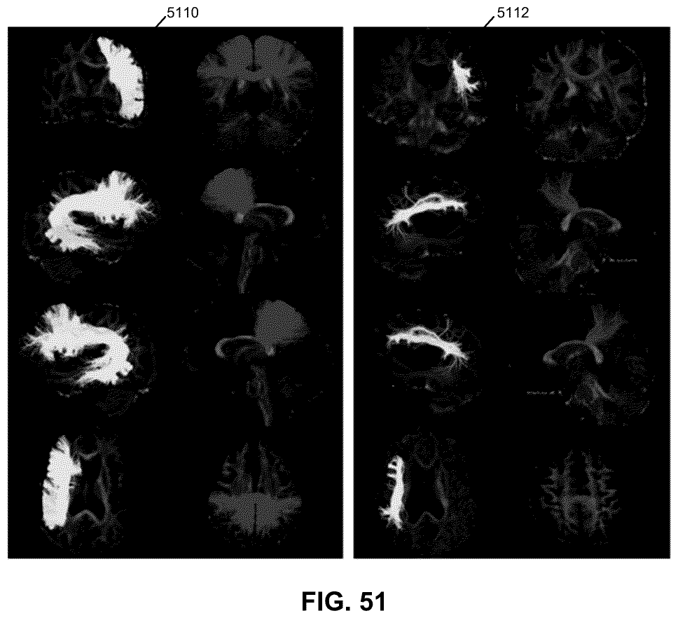

QC of bundle shapes may be performed in a similar manner as for the brain-segmentation maps. Notably, the bundles may be flagged as being too large or too small compared to a predetermined reference size (which varies from one bundle to the next, as discussed further below with reference to Table 3), or as being populated by too-few brain fibers. Moreover, anomalies may also be detected. This may occur when a bundle does not connect the correct gray matter regions, or when a bundle includes fibers with anatomically implausible shapes. This is shown in FIG. 51, which presents an image illustrating examples of correct bundles 5110 (such as the arcuate fasciculus bundle and the corpus callosum_6 bundle) and incorrect (too small) bundles 5112. In some embodiments, a given bundle may be compared to those from a reference brain atlas in order to detect shape anomalies. Alternatively or additionally, a supervised machine-learning model may be trained to differentiate between normal and abnormal bundle shapes. Note that abnormal bundles may also be detected using longitudinal analysis. In these embodiments, the shape, size and position of each bundle should be roughly the same during a study. Therefore, a deviation from this expected trend may indicate the presence of an anomaly.

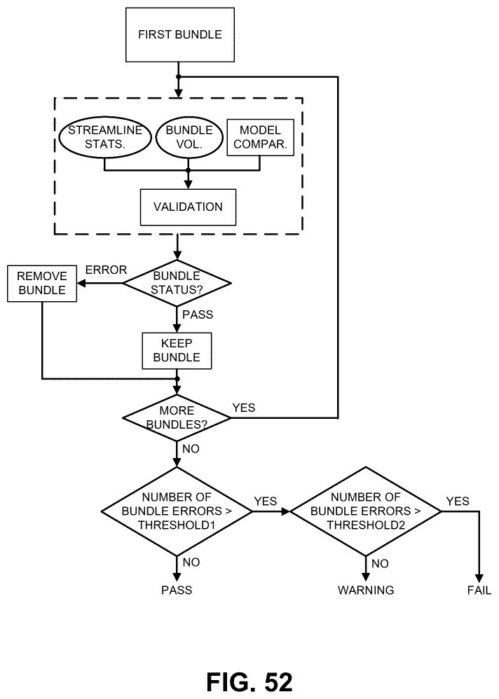

The QC of bundle shapes may result in the rejection of a subset of bundles. As shown in FIG. 52, which presents a drawing illustrating an example of bundle QC in the automatic QC method of FIG. 45, when too many bundles are rejected (e.g. rejection of more than 50% of all bundles), the medical data may also be rejected. However, when fewer bundles are rejected (e.g. between 20% and 50% of all bundles), a warning flag may be raised.

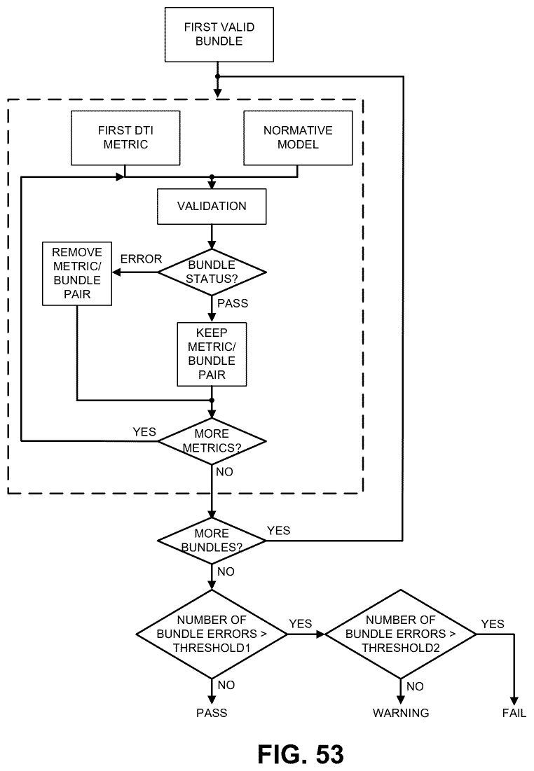

Additionally, QC may be performed on bundle-wise tractometry by evaluating diffusion MRI metrics along each white matter bundle using summary statistics. These metrics may include diffusion tensor imaging (DTI) measures, high angular resolution diffusion imaging (HARDI) metrics, NODDI metrics, or values derived from the orientation distribution function (ODF) and fiber ODF (fODF). For each bundle, statistics, such as the mean, standard deviation or higher-order moments, may be computed across all voxels or fixels. As illustrated in FIG. 53, which presents a drawing illustrating an example of bundle tractometry QC in the automatic QC method of FIG. 45, anomalies can be identified when these statistics deviate from expected patterns. In such cases, the corresponding bundle-metric pair may be excluded. When too many of such pairs have been removed (e.g. more than 25), the medical data may be rejected.

Furthermore, bundle-wise tractometry anomalies may be detected when the values of these statistics fall outside a viable range determined by a minimum and maximum threshold. These thresholds may be specific for each bundle-metric pair (e.g., between 0.4 and 0.7 for fractional anisotropy) and may be computed from a normative reference population. This population may account for co-variables that affect these metrics, such as age, sex, and handedness. With a normative reference population, anomalies may be detected using a statistical test, such as: a z-test, a t-test, an ANOVA test, a Mann-Withney U-test, a Wilcoxon test, a Mann-Whitney Test, a Moods Median test and/or a Kruskal-Wallis test.

In some embodiments, the set of validation operations validates different processes or information extracted from input medical data at each operation in a pipeline. The result of these operations may provide a status corresponding to pass, if no error is detected, or otherwise, fail. In some cases, when a small enough error is detected, a warning flag is raised. As shown in FIG. 45, when the status is fail, the input medical data may be rejected. Furthermore, when a large number of warning flags have been raised (e.g. more than two or three), the input medical data may also be rejected.

We now describe an example of the QC techniques. During validation of metadata, validation may be performed on metadata specific to the acquired images (such as dMRI, T₁, T₂, rev-b0, MTR, ihMT, etc.) and the associated MRI scanner. This metadata may include: the field strength of the MRI scanner; the echo time; the repetition time (TR); the 3D voxel resolution (resSₓ, resS_{y}, resS_{z}); and/or the phase-encoding direction. Then, an error may be asserted when one (or more) of these criteria is met: the field strength is, e.g., different from 1.5 T or 3 T; the voxel resolution (resSₓ, resS_{y}, or resS_{z}) is, e.g., outside of a 0.75-4 mm range; the TE is, e.g., outside of a 50-150 ms range; the TR is, e.g., outside of a 2,000-12,000 ms range; and/or a phase-encoding direction is, e.g., not j or −j. In some embodiments, a QC check verifies that the resolution does exceed a certain anisotropy. For example, let resSₓ, resS_{y}, resS_{z} be the image resolution along the x, y and z axes. An error may be asserted when the ratio between the minimum and maximum value is below 0.5, or

$$\frac{min ( {resS}_{x} , {resS}_{y} , {resS}_{z} )}{max ( {resS}_{x} , {resS}_{y} , {resS}_{z} )} < 0.5 .$$

Moreover, when performing QC on a diffusion gradient, validation may be performed on the b-values and the b-vectors. An error may be asserted when one or more of the following criteria is met: no b0 MR image is available (e.g., one for which the b-value equals 0); for a DTI acquisition, less than, e.g., six noncollinear b-vectors are available; for an HARDI acquisition, less than, e.g., 15 noncollinear b-vectors are available per shell; and/or when the b-vector table exhibits unintentional flips following a fiber coherence index technique. Note that an error may also be asserted when the gradient distribution is non-uniform and exhibits a high discrepancy. This may be performed with the Delaunay triangulation technique described previously. In this case, an error may be asserted when a ratio between the standard deviation and the mean of the triangle areas is greater than, e.g., 0.5.

Furthermore, when performing QC on dMRI artifact correction, an error may be asserted when motion artifacts, eddy-current artifacts and/or slice drop artifacts are detected. This typically occurs when a patient moves their head during image acquisition. When the head motion is too large (see, e.g., Table 2 for exemplary values), this creates artifacts (such as blur) that cannot be compensated for and, thus, may cause processing errors. Note that the approach for measuring motion artifacts may be based at least in part on a nonparametric framework.

Note that the pass or fail criterion in this validation operation may be based at least in part on: head-motion values, eddy-current distortions and/or the detected outlier slices. For example, let (V₁, V₂, . . . , V_{N}) be the N diffusion MRI volume acquired along each b-vector (where N is a non-zero integer). Head motion may be estimated between different pairs of MRI volume Vᵢ and Vⱼ and aggregated in, e.g., seven artifact measures:
Average Absolute Motion: average motion measured between volume V₁ and the other volumes Vᵢ for i=2, . . . , N.
Average Relative Motion: average motion measured between consecutive volumes Vᵢ and Vᵢ₋₁ for i=2, . . . , N.
Rotation: the average rotations between volume V₁ and the other volumes Vᵢ for i=2, . . . , N.
Translation: average translations measured between V₁ and the other volumes Vᵢ for i=2, . . . , N.
Eddy-Current Distortions: using the eddy current-induced off-resonance field model with a low-order polynomial, the standard deviation of the three coefficients of the first-order terms across the whole acquisition.
Ratio of Outliers: a ratio between the total number of detected outlier slices and the total number of slices. A slice may be considered an outlier when the average signal intensity is at least, e.g., four standard deviations below its expected intensity.
Standard Deviation of Outliers: the standard deviation of the signal intensity of a slice detected as an outlier.
   A failure during the set of validation operations may occur when one or more of these measures is above the thresholds shown in Table 2, which provides examples of dMRI artifact correction QC artifact measures and associated thresholds above which an error is asserted. Note that a warning flag can be raised each time a measure in Table 2 is between 90% and 100% of the maximum threshold.

TABLE 2

| Artifact Measure | Maximum Threshold |
|---|---|
| Average Absolute Motion | 1.35 |
| Average Relative Motion | 0.75 |
| Rotation (x, y, z) | ±0.015 |
| Translation (x, y, z) | ±0.7 |
| Eddy Current Distortions (x, y) | 0.2 |
| Ratio of Outliers | 0.006 |
| Standard Deviation of Outliers | 1.5 |

Additionally, during QC on brain-tissue segmentation, the validation may be performed on a mask corresponding to the brain segmentation (which is sometimes referred to as a ‘skull map). In principle, the brain-tissue segmentation may only include white matter, gray matter and cerebrospinal fluid. A fail status may occur when: holes are detected in the mask; and/or the volume of the brain b0 segmentation is outside, e.g., of the range 1,330,000-2,100,000 mm³. A warning flag may be raised when the detected holes are smaller than 5 mm³ and/or when the volume of the brain b0 segmentation is between 1,330,000 and 1,430,000 mm³ or between 2,000,000 and 2,100,000 mm³.

Moreover, during QC on dMRI processing, the automatic validation operation may include validating the extracted diffusion MRI metrics by ensuring that their values are within anatomically plausible boundaries. For example, for the fractional anisotropy, the mean diffusivity, the radial diffusivity, D_{trace} and/or the ADC DTI metrics, an error may be asserted when: the average fractional anisotropy in the white matter of a subject is, e.g., negative or above 1.0; the average mean diffusivity in the white matter of a subject is, e.g., negative or above 0.003; the average radial diffusivity in the white matter of a subject is, e.g., negative or above 0.003; the average D_{trace} in the white matter of a subject is, e.g., negative or above 0.006; and/or the average exponential ADC in the white matter of a subject is, e.g., negative or above 3.0. For the ODF model, note that an automatic check may apply a fail status if the maximum signal value in a dMRI volume is, e.g., below 0.00001, which, otherwise, may corrupt the ODF estimation. Similarly, the fODF computation may include an FRF. The FRF may include four parameters: three may correspond to a low-pass filter and the fourth may be the estimated minimum mean b0 signal in single fiber voxels. In this case, when it is approximated at a signal, e.g., below 900, a fail status may be asserted or applied. Note that, in all cases, a warning flag may be raised when the extracted diffusion MRI metric is 5% below a maximum threshold or 5% above a minimum threshold.

Furthermore, during QC on bundles determined from the images using a tractography and bundling techniques or an atlas registration technique or a machine learning segmentation method, bundle-specific thresholds may be determined in order to detect invalid reconstructed bundles. For example, a bundle-specific threshold may be determined based at least in part on: the number of streamlines, the maximum length of the streamlines, the bundle volume and/or the average overlap volume with bundles from five models. These models may include those used in RBx in the MNI space. Note that the application of the transformation from MNI space to a subject space on the model bundles may make it possible to compare the volumes. Table 3 shows examples of the thresholds for different bundles in the RBx atlas. Notably, a failure may be asserted when a bundle metric exceeds or is greater than a given threshold.

TABLE 3

| | | | Overlap | Maximum |
|---|---|---|---|---|
| | Count | Volume | Volume | Length |
| Bundle | Threshold | Threshold | Threshold | Threshold |
| AF | 100 | 5000 | 0.1 | 100 |
| CC_1 | 50 | 1000 | 0.05 | 60 |
| CC_2a | 100 | 5000 | 0.1 | 90 |
| CC_2b | 100 | 5000 | 0.1 | 90 |
| CC_3 | 100 | 5000 | 0.1 | 90 |
| CC_4 | 100 | 5000 | 0.1 | 90 |
| CC_5 | 100 | 5000 | 0.1 | 90 |
| CC_6 | 100 | 5000 | 0.1 | 100 |
| CC_7 | 100 | 5000 | 0.1 | 130 |
| CG | 100 | 5000 | 0.1 | 120 |
| CR | 100 | 20000 | 0.1 | 100 |
| CST | 100 | 15000 | 0.1 | 100 |
| ICP | 100 | 5000 | 0.1 | 72 |
| IFOF | 100 | 5000 | 0.1 | 150 |
| ILF | 100 | 5000 | 0.1 | 110 |
| MCP | 100 | 5000 | 0.1 | 100 |
| OR | 100 | 5000 | 0.1 | 100 |
| SLF_1 | 50 | 3000 | 0.05 | 110 |
| SLF_2 | 100 | 5000 | 0.1 | 110 |
| SLF_3 | 100 | 5000 | 0.1 | 110 |
| AF | 100 | 5000 | 0.1 | 100 |
| CC_1 | 50 | 1000 | 0.05 | 60 |
| CC_2a | 100 | 5000 | 0.1 | 90 |
| CC_2b | 100 | 5000 | 0.1 | 90 |
| CC_3 | 100 | 5000 | 0.1 | 90 |
| CC_4 | 100 | 5000 | 0.1 | 90 |
| CC_5 | 100 | 5000 | 0.1 | 90 |
| UF | 100 | 5000 | 0.1 | 90 |
| PYT | 100 | 15000 | 0.1 | 100 |
| FX | 50 | 1000 | 0.05 | 80 |
| FAT | 100 | 5000 | 0.10 | 80 |

Additionally, each bundle that fails the QC operation may be removed. When more than 50% of all bundles have been removed, the medical image data may also be rejected. However, when between 20% and 50% of all bundles have been removed, a warning flag may be raised.

Moreover, as part of the QC process for bundle-wise tractometry, the mean value of each diffusion MRI metric may be computed along each white matter bundle of a given subject. This includes, but is not limited to, fractional anisotropy (FA), mean diffusivity (MD), axial diffusivity (AD), the three eigenvalues of the diffusion tensor, free water (FW), and apparent fiber density (AFD). When the computed mean deviates from the normative population's mean by more than 4 standard deviations, the corresponding bundle-metric pair may be excluded. When more than 20 such pairs are rejected, a fatal error is triggered, and the medical imaging data may be discarded. The normative model for each bundle-metric pair may be constructed using the mean and standard deviation of the corresponding metric, measured over a reference population suffering from no known neurological disease of at least 100 individuals matched by sex and within a ±10-year age window of the given subject.

In some embodiments, the automated QC facilitated by the analysis and QC techniques allows large-scale processing of diffusion MR images. Often, diffusion MR images are processed by laboratories in academia. These datasets are often restricted or modest in size and are typically at least partially manually processed. In contrast, the disclosed analysis and QC techniques allow vast datasets to be quality controlled and processed at scale. For example, using the disclosed analysis and QC techniques, 36,465 diffusion MR images were processed over a few months, which is roughly two orders of magnitude larger than the datasets processed by academic laboratories. From these processing results, 425 MR images failed the automated QC testing. After cautious verification, 100% of these cases appear to be truly erroneous. Moreover, 2,000 MR images that passed the automated QC were manually verified, which confirmed that 100% of these cases were true positives. Similarly, client clinics may send a steady stream of diffusion MR images for processing. As a rule of thumb, each clinic can send up to 10 diffusion MR images for processing every day. Furthermore, teleradiologists working with several clinics can send up to 20 diffusion MR images for processing every day. Once again, using the disclosed analysis and QC techniques, 1 to 5% of the diffusion MR images result in processing failures, all of which may be detected using the automated QC. Given that a manual QC verification can take up to 30 minutes per diffusion MR image, the quality of such a constant stream of data can only be assessed using an automatic technique, such as the disclosed analysis and QC techniques.

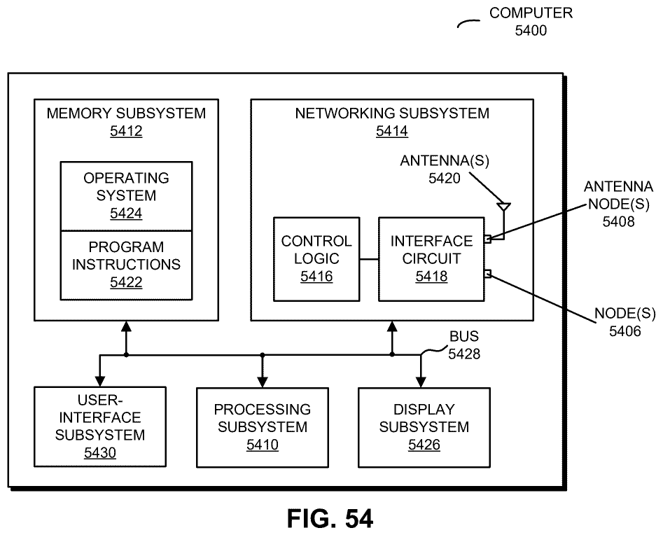

We now describe embodiments of a computer, which may perform at least some of the operations in the analysis and QC techniques. FIG. 54 presents a block diagram illustrating an example of a computer 5400, e.g., in a computer system (such as computer system 1000 in FIG. 10), in accordance with some embodiments. For example, computer 5400 may include: one of computers 1010. This computer may include processing subsystem 5410, memory subsystem 5412, and networking subsystem 5414. Processing subsystem 5410 includes one or more devices configured to perform computational operations. For example, processing subsystem 5410 can include one or more microprocessors, ASICs, microcontrollers, programmable-logic devices, GPUs and/or one or more DSPs. Note that a given component in processing subsystem 5410 are sometimes referred to as a ‘computation device’.

Memory subsystem 5412 includes one or more devices for storing data and/or instructions for processing subsystem 5410 and networking subsystem 5414. For example, memory subsystem 5412 can include dynamic random access memory (DRAM), static random access memory (SRAM), and/or other types of memory. In some embodiments, instructions for processing subsystem 5410 in memory subsystem 5412 include: program instructions or sets of instructions (such as program instructions 5422 or operating system 5424), which may be executed by processing subsystem 5410. Note that the one or more computer programs or program instructions may constitute a computer-program mechanism. Moreover, instructions in the various program instructions in memory subsystem 5412 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 5410.

In addition, memory subsystem 5412 can include mechanisms for controlling access to the memory. In some embodiments, memory subsystem 5412 includes a memory hierarchy that comprises one or more caches coupled to a memory in computer 5400. In some of these embodiments, one or more of the caches is located in processing subsystem 5410.

In some embodiments, memory subsystem 5412 is coupled to one or more high-capacity mass-storage devices (not shown). For example, memory subsystem 5412 can be coupled to a magnetic or optical drive, a solid-state drive, or another type of mass-storage device. In these embodiments, memory subsystem 5412 can be used by computer 5400 as fast-access storage for often-used data, while the mass-storage device is used to store less frequently used data.

Networking subsystem 5414 includes one or more devices configured to couple to and communicate on a wired and/or wireless network (i.e., to perform network operations), including: control logic 5416, an interface circuit 5418 and one or more antennas 5420 (or antenna elements). (While FIG. 54 includes one or more antennas 5420, in some embodiments computer 5400 includes one or more nodes, such as antenna nodes 5408, e.g., a metal pad or a connector, which can be coupled to the one or more antennas 5420, or nodes 5406, which can be coupled to a wired or optical connection or link. Thus, computer 5400 may or may not include the one or more antennas 5420. Note that the one or more nodes 5406 and/or antenna nodes 5408 may constitute input(s) to and/or output(s) from computer 5400.) For example, networking subsystem 5414 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, and/or another networking system.

Networking subsystem 5414 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, computer 5400 may use the mechanisms in networking subsystem 5414 for performing simple wireless communication between electronic devices, e.g., transmitting advertising or beacon frames and/or scanning for advertising frames transmitted by other electronic devices.

Within computer 5400, processing subsystem 5410, memory subsystem 5412, and networking subsystem 5414 are coupled together using bus 5428. Bus 5428 may include an electrical, optical, and/or electro-optical connection that the subsystems can use to communicate commands and data among one another. Although only one bus 5428 is shown for clarity, different embodiments can include a different number or configuration of electrical, optical, and/or electro-optical connections among the subsystems.

In some embodiments, computer 5400 includes a display subsystem 5426 for displaying information on a display, which may include a display driver and the display, such as a liquid-crystal display, a multi-touch touchscreen, etc. Moreover, computer 5400 may include a user-interface subsystem 5430, such as: a mouse, a keyboard, a trackpad, a stylus, a voice-recognition interface, and/or another human-machine interface.

Computer 5400 can be (or can be included in) any electronic device with at least one network interface. For example, computer 5400 can be (or can be included in): a desktop computer, a laptop computer, a subnotebook/netbook, a server, a supercomputer, a tablet computer, a smartphone, a cellular telephone, a consumer-electronic device, a portable computing device, communication equipment, and/or another electronic device.

Although specific components are used to describe computer 5400, in alternative embodiments, different components and/or subsystems may be present in computer 5400. For example, computer 5400 may include one or more additional processing subsystems, memory subsystems, networking subsystems, and/or display subsystems. Additionally, one or more of the subsystems may not be present in computer 5400. Moreover, in some embodiments, computer 5400 may include one or more additional subsystems that are not shown in FIG. 54. Also, although separate subsystems are shown in FIG. 54, in some embodiments some or all of a given subsystem or component can be integrated into one or more of the other subsystems or component(s) in computer 5400. For example, in some embodiments program instructions 5422 are included in operating system 5424 and/or control logic 5416 is included in interface circuit 5418.

Moreover, the circuits and components in computer 5400 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 5414 and/or computer 5400. The integrated circuit may include hardware and/or software mechanisms that are used for transmitting signals from computer 5400 and receiving signals at computer 5400 from other electronic devices. Aside from the mechanisms herein described, radios are generally known in the art and hence are not described in detail. In general, networking subsystem 5414 and/or the integrated circuit may include one or more radios.

In some embodiments, an output of a process for designing the integrated circuit, or a portion of the integrated circuit, which includes one or more of the circuits described herein may be a computer-readable medium such as, for example, a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the analysis and QC techniques may be implemented using program instructions 5422, operating system 5424 (such as a driver for interface circuit 5418) or in firmware in interface circuit 5418. Thus, the analysis and QC techniques may be implemented at runtime of program instructions 5422. Alternatively or additionally, at least some of the operations in the analysis and QC techniques may be implemented in a physical layer, such as hardware in interface circuit 5418.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the analysis and QC techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
a computation device; and
memory configured to store program instructions, wherein, when executed by the computation device, the program instructions cause the computer system to perform one or more operations comprising:
automatically performing a set of validation operations on images associated with diffusion magnetic resonance imaging (dMRI) and structural magnetic resonance imaging (MRI), wherein, when one or more of the validation operations fails, or when a number of warnings exceed a predefined amount, the images are rejected, and
wherein the set of validation operations comprise: performing quality control (QC) on brain-tissue segmentation; performing QC on dMRI processing; performing QC on bundles determined from the images using a bundling or a segmentation technique; and performing QC on bundle-wise tractometry.

2. The computer system of claim 1, wherein the QC on the brain-tissue segmentation comprises validating one or more of: a volume; a shape; a position; a comparison with a reference-atlas coordinate system; or detecting holes in a mask.

3. The computer system of claim 1, wherein the QC on the dMRI processing comprises validating one or more of: a range of diffusion tensor imaging (DTI) metrics; a range of high angular resolution diffusion imaging (HARDI) metrics; a range of Neurite Orientation Dispersion and Density Imaging (NODDI) metrics; or ranges of dMRI signals for an orientation distribution function (ODF) and a fiber ODF (fODF); or
wherein the QC on the bundle-wise tractometry comprises validating one or more of: a range of diffusion tensor imaging (DTI) metrics; a range of high angular resolution diffusion imaging (HARDI) metrics; a range of Neurite Orientation Dispersion and Density Imaging (NODDI) metrics; or ranges of dMRI signals for an orientation distribution function (ODF) and a fiber ODF (fODF) measured along each bundle and compared to a normative reference.

4. The computer system of claim 1, wherein the QC on the bundles comprises validating one or more of: streamline statistics; bundle volume; bundle shape; or model comparisons.

5. The computer system of claim 1, wherein the set of validation operations comprises: validating metadata associated with the images; performing QC on a diffusion gradient; and performing QC on dMRI artifact correction.

6. The computer system of claim 5, wherein the diffusion gradient comprises: b-values, gradient sampling and a gradient configuration.

7. The computer system of claim 5, wherein the dMRI artifact correction comprise one or more of: motion detection, eddy-current-distribution detection, or slice-outlier detection.

8. The computer system of claim 7, wherein motion detection, comprises measuring the overlap between the brain segmentation maps obtained on two or more MRI and/or dMRI images.

9. The computer system of claim 1, wherein the set of validation operations are implemented using a feed-forward pipeline.

10. The computer system of claim 1, wherein the brain-tissue segmentation comprises: segmenting the gray matter, gray matter sub-regions, the white matter, white matter sub-regions, the cerebrospinal fluid, and/or deep nuclei regions; and removing voxels that are not associated with brain tissue.

11. The computer system of claim 10, wherein the computer system uses the images associated with the structural MRI to extract the brain tissue.

12. The computer system of claim 10, wherein the computer system uses a remainder of the images associated with the dMRI to determine local models for voxels; and
wherein the local models indicate directions of water diffusion.

13. The computer system of claim 12, wherein the operations comprise one or more of: computing diffusion metrics based at least in part on the local models, recovering tracts in the brain tissue, or connecting grey-matter regions using the bundles.

14. The computer system of claim 1, wherein the validation operations comprise a pretrained neural network, and the operations comprise determining the bundles using the pretrained neural network.

15. The computer system of claim 1, wherein a given validation operation comprises comparing the given validation operation with a given threshold associated with the given validation operation.

16. A non-transitory computer-readable storage medium for use in conjunction with a computer system, the computer-readable storage medium configured to store program instructions that, when executed by the computer system, causes the computer system to perform one or more operations comprising:
obtaining images associated with diffusion magnetic resonance imaging (dMRI) and structural magnetic resonance imaging (MRI); and
automatically performing a set of validation operations on the images, wherein, when one or more of the validation operations fails, or when a number of warnings exceed a predefined amount, the images are rejected, and
wherein the set of validation operations comprise: performing quality control (QC) on brain-tissue segmentation; performing QC on dMRI processing; performing QC on bundles determined from the images using a bundling or a segmentation technique; and performing QC on bundle-wise tractometry.

17. The non-transitory computer-readable storage medium of claim 16, wherein the QC on the brain-tissue segmentation comprises validating one or more of: a volume; a shape; a position; a comparison with a reference-atlas coordinate system; or detecting holes in a mask.

18. A method for automatically performing a set of validation operations, comprising:
by a computer system:
obtaining images associated with diffusion magnetic resonance imaging (dMRI) and structural magnetic resonance imaging (MRI); and
automatically performing the set of validation operations on the images, wherein, when one or more of the validation operations fails, or when a number of warnings exceed a predefined amount, the images are rejected, and
wherein the set of validation operations comprise: performing quality control (QC) on brain-tissue segmentation; performing QC on dMRI processing; performing QC on bundles determined from the images using a bundling or a segmentation technique; and performing QC on bundle-wise tractometry.

19. The method of claim 18, wherein the QC on the brain-tissue segmentation comprises validating one or more of: a volume; a shape; a position; a comparison with a reference-atlas coordinate system; or detecting holes in a mask.

20. The method of claim 18, wherein the QC on the dMRI processing comprises validating one or more of: a range of diffusion tensor imaging (DTI) metrics; a range of high angular resolution diffusion imaging (HARDI) metrics; a range of Neurite Orientation Dispersion and Density Imaging (NODDI) metrics; or ranges of dMRI signals for an orientation distribution function (ODF) and a fiber ODF (fODF).
